(19) United States
(12) Patent Application Publication
(10) Pub. No.: US 2013/0143840 A1
(43) Pub. Date: Jun. 6, 2013

(54) HEPARAN SULFATE REPLACEMENT THERAPY

(71) Applicants: Christopher Richard Parish, Campbell (AU); Charmaine Simeonovic, Higgins (AU); Craig Geoffrey Freeman, Rivett (AU)

(72) Inventors: Christopher Richard Parish, Campbell (AU); Charmaine Simeonovic, Higgins (AU); Craig Geoffrey Freeman, Rivett (AU)

(21) Appl. No.: 13/634,254

(22) PCT Filed: Mar. 11, 2011
(86) PCT No.: PCT/AU11/00284

§ 371 Date: Feb. 4, 2013

Related U.S. Application Data
(60) Provisional application No. 61/313,624, filed on Mar. 12, 2010.

(51) Int. Cl. A61K 31/737

(52) U.S. Cl. CPC A61K 31/737
USPC 514/56

(57) ABSTRACT

The present invention relates to a method for inhibiting oxidative damage of islet beta cells in vivo in a subject by administering to the subject a therapeutically effective amount of heparan sulfate capable of protecting islet beta cells from reactive oxygen species or in vitro by exposing isolated islet beta cells, prior to transplantation, to a concentration of heparan sulfate that protects them from reactive oxygen species.

### TECHNICAL FIELD

The present invention relates to the use of heparan sulfate and mimetics thereof in the treatment of Type I or Type II diabetes. In particular the invention relates to the preservation of beta-cell function and treatment and prevention of pancreatic islet rejection after transplantation.

### BACKGROUND

Type I and Type II diabetes have different aetiologies but both diseases are characterised by compromised production of insulin by beta cells in the pancreatic islets of Langerhans. In Type I diabetes the islet beta cells are destroyed by the immune system as a result of an autoimmune response against islet auto-antigens. In Type II diabetes surviving islet beta cells are unable to produce sufficient insulin to compensate for the “insulin resistance” of peripheral tissues.

Transplantation of pancreatic islets is a therapeutic approach for treating diabetes. Use of immunosuppressive drugs is required to prevent the rejection of islet transplants which limits islet transplantation to adult subjects whose diabetes has been difficult to control. In the long term, islet function is eventually lost and insulin therapy is again required. This graft failure is most likely due to toxicity of the immunosuppressive drugs used to prevent immunological rejection of the transplant and/or to recurrence of autoimmune disease. Isolation of functional islets is crucial if successful transplantation is to occur, irrespective of the problems associated with the recipient's immune response against the allograft.

The inventors have shown that preservation of intra-islet heparan sulfate during islet isolation is an important factor in ensuring that normal islet function is retained, the intra-islet heparan sulfate rendering the islet beta cells resistant to reactive oxygen species (ROS). Accordingly, there is a need to counter the loss of islet heparan sulfate in Type I and Type II diabetes which is associated with disease progression as well as protecting islet beta cells from heparan sulfate loss during their isolation for transplantation.

### SUMMARY

According to a first aspect, there is provided a method for inhibiting oxidative damage of islet beta cells in a subject comprising administering to the subject a therapeutically effective amount of heparan sulfate.

According to a second aspect, there is provided a method for inhibiting oxidative damage of islet beta cells comprising contacting said beta cells with heparan sulfate.

According to a third aspect, there is provided a method of treating diabetes comprising administering to a subject in need thereof a therapeutically effective amount of heparan sulfate.

In one embodiment, the diabetes is Type-I or Type-II diabetes.

According to a fourth aspect, there is provided a method of treating an autoimmune condition comprising administering to a subject in need thereof a therapeutically effective amount of heparan sulfate.

In one embodiment, the autoimmune condition may be selected from the group comprising Type 1 diabetic insulitis, rejection of pancreatic islet transplant or a combination thereof.

According to a fifth aspect, there is provided a method of preserving beta-cell function comprising administering to a subject in need thereof a therapeutically effective amount of heparan sulfate.

The beta-cell may be in situ or within a transplanted islet.

According to a sixth aspect, there is provided a method of preserving beta-cell function in isolated islets comprising pretreating the islets with a therapeutically effective amount of heparan sulfate prior to transplantation into a patient.

According to a seventh aspect, there is provided a method of treating or preventing the rejection of a transplant comprising administering to a subject in need thereof a therapeutically effective amount of heparan sulfate.

In one embodiment the transplant is a pancreatic islet transplant.

According to a eighth aspect, there is provided a method for reducing the level of immunosuppressive therapy associated with transplantation comprising administering to a subject in need thereof a therapeutically effective amount of heparan sulfate.

In one embodiment the transplantation is pancreatic islet transplantation.

According to a ninth aspect, there is provided a method for preserving endogenous heparan sulfate comprising administering to a subject a therapeutically effective amount of heparan sulfate. In one embodiment the method further comprises administration of a reactive oxygen species scavenger in combination with the heparan sulfate. The reactive oxygen species scavenger may be selected from the group comprising melatonin, vitamin E, vitamin C, methionine, taurine, Superoxide dismutase (SOD), catalase (CAT), and glutathione peroxidase (GPX), L-ergothioneine N-Acetyl Cysteine (NAC), vitamin A, beta-carotene, retinol, catechins, epicatechins, epigallocatechin-3-gallate, flavonoids, L-ergothioneine, idebenone, selenium, heme oxygenase-1, reduced glutathione (GSH), resveratrol, Tiron (4,5-dihydroxy-1,3-benzenedisulfonic acid), Tempol (4-hydroxy-2,2,6,6-tetramethylpiperydine-1-oxyl), dimethylthiourea (DMTU) and butylated hydroxyanisole (BHA).

According to an tenth aspect, there is provided use of heparan sulfate for the preparation of a medicament for preserving beta-cell function.

According to a eleventh aspect, there is provided use of heparan sulfate for the preparation of a medicament for treatment of diabetes.

In one embodiment, the diabetes is Type-I or Type-II diabetes

According to a twelfth aspect, there is provided use of heparan sulfate for the preparation of a medicament for treatment of transplant rejection.

In one embodiment the transplantation is a pancreatic islet transplant.

According to a thirteenth aspect, there is provided use of heparan sulfate for the preparation of a medicament for inhibiting the rejection of a transplant in a subject.

According to a fourteenth aspect, there is provided use of heparan sulfate for the preparation of a medicament for reducing the level of immunosuppressive therapy associated with transplantation.

In one embodiment the medicament further comprises a reactive oxygen species scavenger in combination with the heparan sulfate. The reactive oxygen species scavenger may be selected from the group comprising melatonin, vitamin E, vitamin C, methionine; taurine, Superoxide dismutase (SOD), catalase (CAT), glutathione peroxidase (GPX), L-ergothioneine N-Acetyl Cysteine (NAC), vitamin A, beta-carotene, retinol, catechins, epicatechins, epigallocatechin-3-gallate, flavonoids, L-ergothioneine, idebenone, selenium, heme oxygenase-1, reduced glutathione (GSH), resveratrol, Tiron (4,5-dihydroxy-1,3-benzenedisulfonic acid), Tempol (4-hydroxy-2,2,6,6-tetramethylpiperydine-1-oxyl), dimethylthiourea (DMTU) and butylated hydroxyanisole (BHA).

The heparan sulfate may be maltohexaose sulfate. The heparan sulfate may be covalently bound to a molecule to increase the half-life of the heparan sulfate. The covalently bound heparan sulfate may be PEGylated. The covalently bound heparan sulfate may be peroxidolysis-glycol split (3 kDa) heparin.

According to a fifteenth aspect, there is provided heparan sulfate for use in inhibiting oxidative damage of islet beta cells.

According to a sixteenth aspect, there is provided heparan sulfate for use in treatment of diabetes.

In one embodiment, the diabetes is Type-I or Type-II diabetes.

According to a seventeenth aspect, there is provided heparan sulfate for use in preserving beta-cell function.

According to an eighteenth aspect, there is provided heparan sulfate for use in inhibiting the rejection of a transplant in a subject.

According to a nineteenth aspect, there is provided heparan sulfate for use in preserving beta-cell function in isolated islets by pretreating the islets with heparan sulfate prior to transplantation into a patient.

### BRIEF DESCRIPTION OF THE DRAWINGS

A preferred embodiment of the invention will now be described, by way of an example only, with reference to the accompanying drawings wherein:

FIG. 1 shows Alcian blue staining of heparan sulfate in the islet cell mass of a non-diabetic (a) mouse and (b) human islet, in situ in the pancreas.

FIG. 2 shows immunohistochemical staining of the heparan sulfate proteoglycans (a) collagen type XVIII and (b) syndecan-1 in non-diabetic mouse islets in situ in the pancreas. (c) Immuno-histochemical staining for heparan sulfate confirms the localisation of this glycosaminoglycan observed with Alcian blue histochemistry.

FIG. 3 shows by Alcian blue staining, heparan sulfate localisation in islets in the pancreas of (a) db heterozygous (normal phenotype) and (b) Type II diabetic db homozygous mice.

FIG. 4 shows flow cytometry analysis of isolated BALB/c beta cell viability following culture for 2 days either in the absence (Control) or presence of 50 μg/ml of heparin, highly sulfated heparan sulfate (HS^{hi}) or the HS-mimetic PI-88. Viability was assessed by Sytox green uptake or by Calcein-AM (viable and early apoptotic cells) and propidium iodide (PI; dead and apoptotic cells) uptake (lower panels). Counting beads (CB) shown in upper panels were added to the samples prior to staining and FACS analysis to determine the number of beta cells stained.

FIG. 5 shows that heparin, HS^{hi} and PI-88 can protect islet beta cells from culture-induced cell death. Flow cytometry analysis using Sytox green staining shows that the protective effects of heparin, HS^{hi} and PI-88 on beta cell viability is dose dependent. In contrast to HS^{hi} and PI-88, heparin preserved beta cell survival at 5 μg/ml during 2 days of culture.

FIG. 6 shows that co-culture with heparin or highly sulfated HS protects isolated islet beta cells from culture-induced cell death. Absolute number of beta cells and number of dead beta cells in 2 day control cultures and in 2 day cultures containing 50 μg/mL of heparin, HS^{hi} or PI-88 from FIG. 4 (upper panel), the numbers of beta cells being calculated by adding counting beads to the samples prior to staining and flow cytometry analysis (shown at top of upper panels in FIG. 4 as CB).

FIG. 7 shows that co-culture with highly sulfated HS, but not undersulfated HS, protects isolated islet beta cells from culture-induced cell death. Comparison of the ability of HS^{hi} (50 μg/ml) and undersulfated HS(HS^{lo}, 50 μg/ml) to protect beta cells from 2 day culture-induced cell death, as shown by Sytox green uptake (upper panels). Lower panels depict the intracellular insulin content (green histograms) of the different preparations of cultured islet cells. Serum control (red histograms) and islet cell autofluorescence (black histograms) are also shown. 85-88% of the cultured islet cells were insulin⁺ beta cells.

FIG. 8 shows that heparin from both bovine lung and porcine intestinal mucosa protects islet beta cells from culture-induced cell death. (A) Porcine intestinal mucosa (Pore Int Muc) heparin is equally effective as bovine lung (Bov Lung) heparin in protecting beta cells from culture-induced cell death after 2 days of culture. Viability was assessed by Sytox green uptake (upper panels) or by Calcein-AM (viable and early apoptotic cells) and PI (dead and late apoptotic cells) uptake (lower panels). The unbounded region at the top of the dot plots for Sytox green staining represents counting beads (CB) added to the cells prior to staining and flow cytometry analysis. (B) Time course of absolute number of beta cells and number of dead beta cells after 1 h, 1 day or 2 days of culture in the presence or absence of bovine lung heparin (50 μg/ml). Cell numbers were calculated using the counting beads as shown in FIG. 4 (top panels).

FIG. 9 shows uptake of FITC-heparin by islet beta cells. (A) Confocal microscopy of mouse beta cells cultured for 2 days with FITC-labelled heparin (50 μg/ml) demonstrates substantial intracellular uptake of FITC-heparin. (B) Flow cytometry analysis of the beta cells from A revealed FITC-heparin uptake by 89% of the beta cells with 86% of the cultured beta cells being FITC-heparin⁺PI⁻, indicating that the FITC-heparin protected the beta cells from culture-induced cell death.

FIG. 10 shows by Sytox green immunofluorescence staining of freshly isolated BALB/c beta cells (day 0) that they are sensitive to hydrogen peroxide-induced death (59.5% cell death in day 0 controls versus 96.1% cell death after hydrogen peroxide treatment on day 0). Co-culture of beta cells with heparin (50 μg/ml) protects the beta cells from both death in culture (72.5% dead in controls vs 5.3% in treated cell cultures) and following hydrogen peroxide treatment (5.0% cell death).

FIG. 11 shows highly sulfated HS(HS^{hi}) and PI-88 protect islet beta cells from ROS-mediated cell death. (A) Mouse beta cells when cultured for 2 days with HS^{hi} (50 μg/ml) but not when cultured with HS^{lo} (50 μg/ml), were protected from culture-induced and hydrogen peroxide (ROS)-mediated cell death, compared to control cultures. Note that 93% of beta cells, prior to culture, were killed by treatment with H₂O₂ (data not shown). (B) Culturing beta cells with PI-88 (50 μg/ml) for 2 days also protected the beta cells from culture-induced and ROS-mediated cell death.

FIG. 12 shows by Alcian blue staining that in contrast to mouse islets in situ in the pancreas, which stain strongly for heparan sulfate (a), freshly isolated islets show substantial loss of their heparan sulfate (b). In fact, the lower panel shows that the area of intra-islet staining with Alcian blue was significantly higher in BALB/c islets in situ (n=50) than in isolated islets (n=45), as quantified by Image J software with Color Deconvolution plugin (P<0.0001 by Mann-Whitney test).

FIG. 13 shows by Alcian blue histochemistry shows weak localisation of heparan sulfate in isografts of isolated islets at day 3 post-transplant (a). By day 7 (b), the engrafted islets show reconstitution of their intra-islet heparan sulfate.

FIG. 14 shows flow cytometry analysis of the insulin content of isolated beta cells reveals that compared to control beta cells (a), the insulin content of beta cells is substantially increased if the cells are prepared from islets isolated in the presence of 50 μg/ml heparin (b).

FIG. 15 illustrates that treatment of recipient alloxan-induced diabetic C57BL/6J mice (H-2^{b}) with a heparan sulfate mimetic (peroxidolysis-glycol split (3 kDa) heparin derivative; 3×40 mg/kg/day) i.p. prolongs the survival and function of a CBA (H-2^{k}) islet allograft to 15 days (a). In contrast, a saline treated control recipient showed loss of islet allograft survival and function by 9 days. Blue speckled bar represents the normal blood glucose range of healthy mice. Treatment with a heparan sulfate mimetic therefore represents a novel anti-rejection therapy.

FIG. 16 shows by that Alcian blue histochemistry that treatment of pre-diabetic NOD mice with the heparan sulfate mimetic PI-88 (10 mg/kg/day, i.p.) restored the heparan sulfate content of islets with destructive insulitis compared to saline treated control mice which exhibited substantial loss of islet heparan sulfate in the presence of destructive insulitis.

### DEFINITIONS

Certain terms are used herein which shall have the meanings set forth as follows.

As used herein, the term “comprising” means “including principally, but not necessarily solely”. Furthermore, variations of the word “comprising”, such as “comprise” and “comprises”, have correspondingly varied meanings.

Unless the context requires otherwise or specifically stated to the contrary, integers, steps, or elements of the invention recited herein as singular integers, steps or elements clearly encompass both singular and plural forms of the recited integers, steps or elements.

As used herein the terms “treating” and “treatment” refer to any and all uses which remedy a condition or symptoms; prevent the establishment of a condition or disease, or otherwise prevent, hinder, retard, or reverse the progression of a condition or disease or other undesirable symptoms in any way whatsoever.

In the context of this specification the term “therapeutically effective amount” includes within its meaning a sufficient but non-toxic amount of a compound or composition of the invention to provide the desired effect. The exact amount required will vary from subject to subject depending on factors such as the desired effect, the species being treated, the age and general condition of the subject, the severity of the condition being treated, the particular agent being administered, the mode of administration, and so forth. Thus, it is not possible to specify an exact “therapeutically effective amount”. However, for any given case, an appropriate “therapeutically effective amount” may be determined by one of ordinary skill in the art using only routine experimentation.

As used herein the term “reactive oxygen species” or “ROS” refers to molecules or ions formed by the incomplete one-electron reduction of oxygen. These reactive oxygen species include singlet oxygen; superoxides; peroxides; hydroxyl radicals; nitric oxide and hypochlorous acid.

As used herein the term “heparan sulfate” refers to heparan sulfate and molecules capable of mimicking at least one biological function of heparan sulfate.

As used herein, the term “alkyl” includes within its meaning monovalent straight chain or branched chain saturated hydrocarbon radicals having from 1 to 10 carbon atoms, eg, 1, 2, 3, 4, 5, 6, 7, 8, 9, or 10 carbon atoms. For example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, and the like.

As used herein the term “alkylene” includes within its meaning divalent straight chain or branched chain saturated hydrocarbon radicals having from 1 to 10 carbon atoms.

As used herein, the term “alkenyl” includes within its meaning monovalent straight chain or branched hydrocarbon radicals having at least one double bond, and having from 2 to 10 carbon atoms, eg, 2, 3, 4, 5, 6, 7, 8, 9, or 10 carbon atoms. For example, the term alkenyl includes, but is not limited to vinyl, propenyl, 2-methylbutenyl and hexenyl.

As used herein, the term “alkoxy” refers to O-alkyl, where alkyl is as defined above.

As used herein the term “halo” includes within its meaning fluoro, chloro, bromo and iodo.

As used herein, the term “aryl” or “Ar” includes within its meaning monovalent, single, polynuclear, conjugated and fused aromatic hydrocarbon radicals, for example phenyl, naphthyl, anthracenyl, pyrenyl, phenanthracenyl.

As used herein, the term “heteroaryl” includes within its meaning monovalent, single, polynuclear conjugated and fused aromatic radicals having 1 to 15 carbons wherein 1 to 6 atoms are hetero atoms selected from O, N and S.

As used herein the term “arylene” includes within its meaning divalent, single, polynuclear, conjugated and fused aromatic hydrocarbon radicals.

As used herein the term “cyclitol” includes within its meaning cycloalkanes comprising one hydroxyl group on each of three or more ring atoms.

As used herein the term “pseudo sugar” includes within its meaning monosaccharide, disaccharide or oligosaccharide molecules in which one or more of the “saccharide” units do not comprise an oxygen atom.

In the context of the present specification, “low molecular weight anionic glycan mimetic” refers to sugar or saccharide mimetics or analogues or sugar-like compounds having molecular weights less than about 5 kDa.

In the context of the present specification the terms “ring-opened monosaccharide”, “ring-opened disaccharide” and “ring-opened oligosaccharide” refer to the respective saccharide molecules wherein at least one ring is present in the open chain form. The “ring-opened” compound may be for example an alditol or a glycol split, or any other product of complete or partial oxidation and/or reduction of said monosaccharide, disaccharide or oligosaccharide arising from, for example, reactions as are known in the art such as sodium borohydride reduction.

### DETAILED DESCRIPTION

It is to be understood at the outset, that the figures and examples provided herein are to exemplify, and not to limit the invention and its various embodiments.

In accordance with the present invention methods are provided for inhibiting oxidative damage of islet beta cells. The present invention also provides compositions and methods for the treatment of Type I and Type II diabetes, the isolation of islets and the treatment and/or prevention of pancreatic islet rejection after transplantation. The methods generally comprise the use of compositions comprising heparan sulfate. The methods also comprise the use of compositions comprising heparan sulfate together with at least one reactive oxygen species scavenger.

Type I and Type II diabetes have a common pathological feature which is compromised production of insulin by the beta cells in the pancreatic Islets of Langerhans. In the case of Type I diabetes the islet beta cells are destroyed by the immune system as a result of an autoimmune response against islet auto-antigens. In contrast, in Type II diabetes, despite the islet beta cells surviving they are unable to produce sufficient insulin to compensate for the “insulin resistance” of peripheral tissues. Usually Type II diabetes is associated with obesity. Paradoxically only a minority of obese individuals develop diabetes.

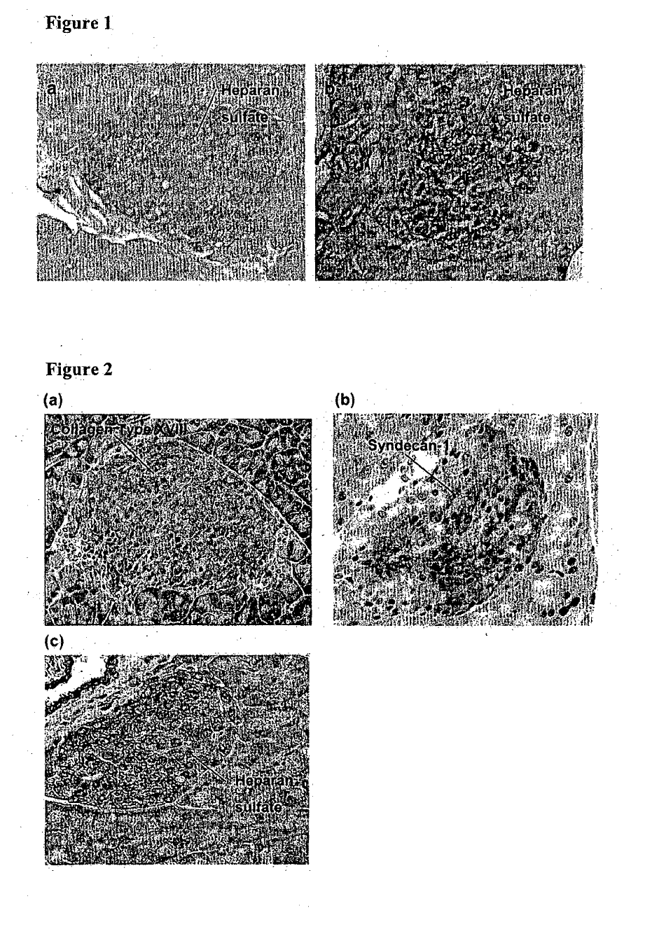

As described herein pancreatic islets, in both mice and humans, contain high levels of the glycosaminoglycan heparan sulfate (FIG. 1) and substantial amounts of collagen XVIII and syndecan-1 (FIGS. 2a and 2b), well known core proteins for heparan sulfate proteoglycans. Such high level expression of heparan sulfate proteoglycans is usually restricted to basement membranes and is not normally present throughout tissues. Furthermore, the islet heparan sulfate is contained within the beta cells themselves rather than being expressed on the beta cell surface and within the extracellular matrix, the normal location of these molecules.

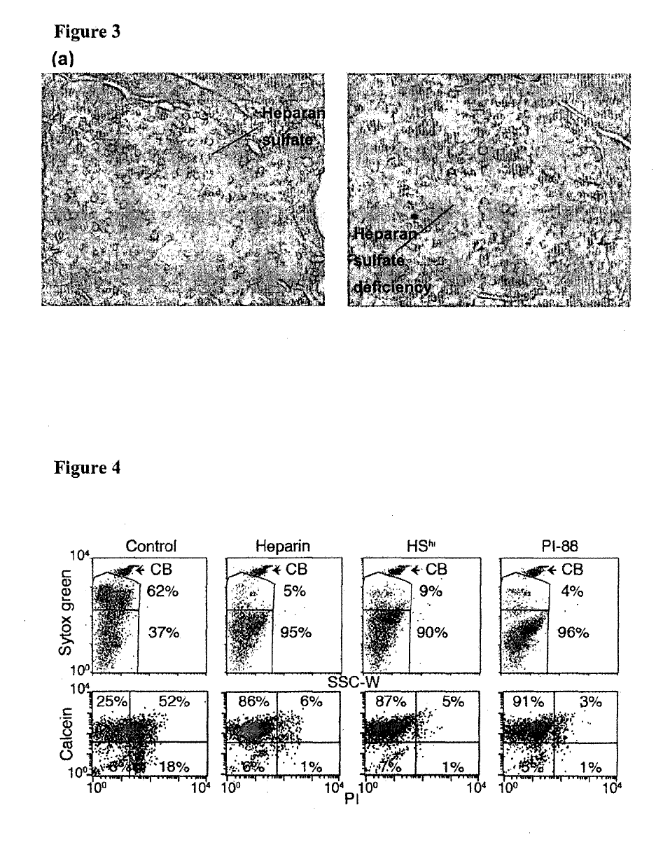

These observations surprisingly suggest that islet heparan sulfate plays a role in beta cell function. A useful animal model for the study of diabetes is the db/db mouse, an obese mouse strain which spontaneously develops Type II diabetes. Islets from diabetic db/db mice contain less heparan sulfate than islets from non-diabetic db heterozygous mice (FIG. 3). During the autoimmune destruction of islets in Type I diabetes a dramatic loss of islet heparan sulfate occurs, a process that is thought to be mediated by the leukocyte-derived endoglycosidase, heparanase (WO 2008/046162). Administration of a heparanase inhibitor substantially reduced the incidence of Type I diabetes in non-obese diabetic (NOD) mice. Collectively, these data surprisingly suggested a common link between Type I and Type II diabetes, where loss of islet heparan sulfate is associated with disease progression.

Heparan Sulfate Protects Islet Beta Cells from Reactive Oxygen Species Induced Cell Death

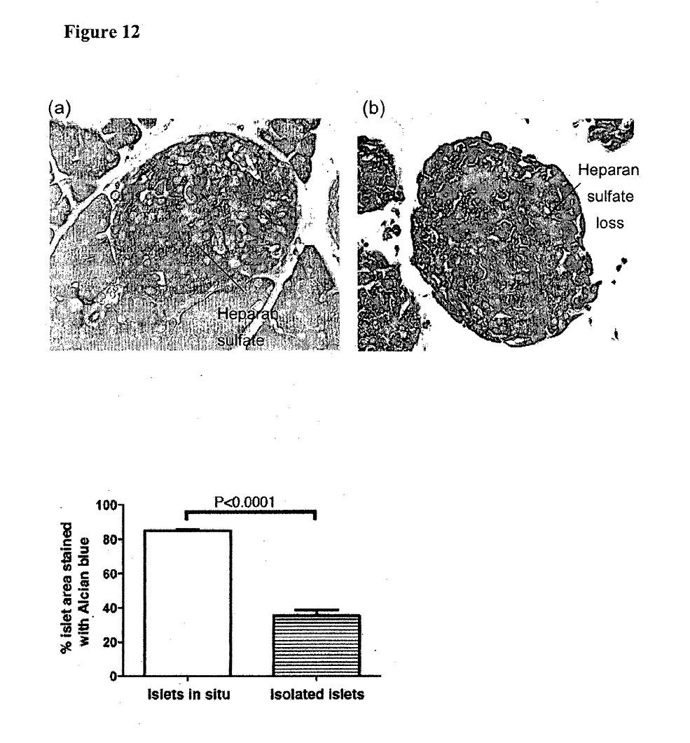

Further support for the concept that heparan sulfate is associated with disease progression was obtained from cultures of isolated islet beta cells (FIGS. 4-9, Table 1) which suggest that loss of islet-associated heparan sulfate during the islet isolation procedure (collagenase digestion and hand-picking) (FIG. 12) results in significantly reduced beta cell survival and that the beta cells can be rescued from dying in culture by providing an exogenous source of highly sulfated heparan sulfate or a range of heparan sulfate derivatives and mimetics, such as heparin from different sources (FIG. 8); glycol split heparin and sulfated oligosaccharides (e.g., PI-88) (FIGS. 4-7 and 9, Table 3).

Cellular metabolism is associated with the production of reactive oxygen species (ROS) which induce oxidative damage to cells, proteins and nucleic acids which can ultimately lead to cell death. Thus, ROS are implicated as signalling molecules that contribute to disease. The generation of ROS is also associated with oxidative stress, apoptosis and necrotic cell death. In the context of transplantation, cell death within implanted islets has deleterious consequences in islet transplantation.

Reactive oxygen species (ROS) include for example superoxide radicals, hydroxyl radicals, nitric oxide, ozone, thiyl radicals, and carbon-centred radicals (e.g., trichloromethyl radical). ROS such as H₂O₂, O₂⁻, .OH and NO, have detrimental effects including inactivation of specific enzymes via oxidation of their co-factors, oxidation of polydesaturated fatty acids in lipids, oxidation of amino acids within proteins and DNA damage.

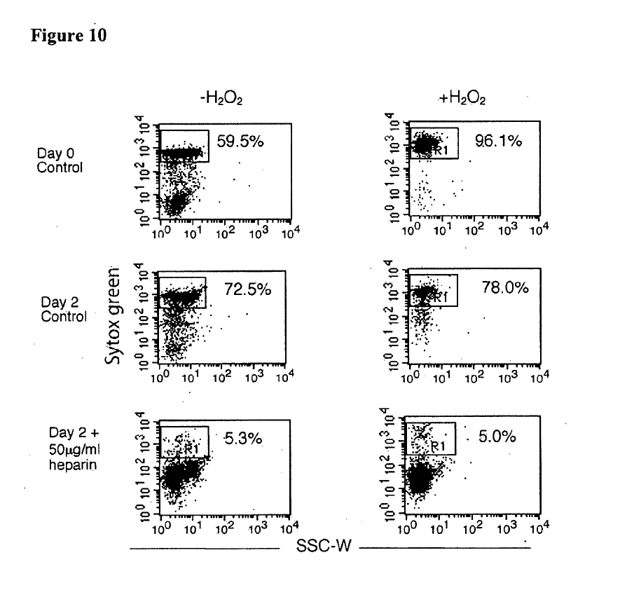

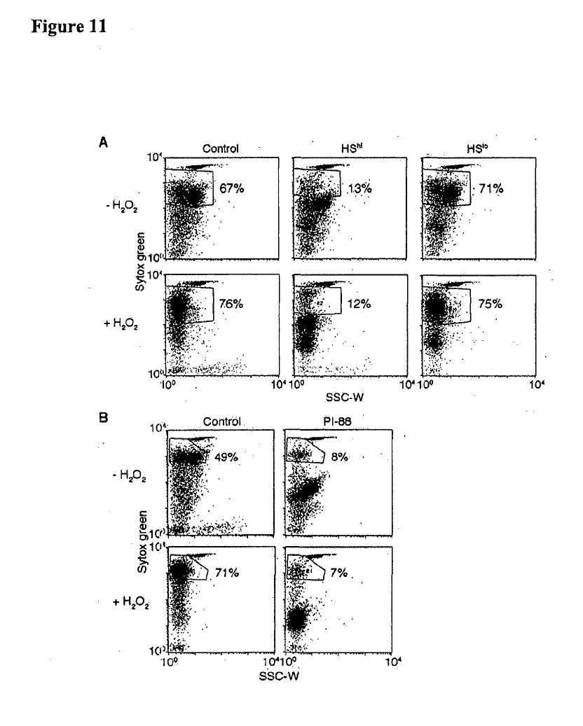

As illustrated in Example 4 and FIGS. 10 and 11 and Tables 2 and 3, the addition of heparin, highly sulfated heparan sulfate, sulfated oligosaccharides (e.g., PI-88), heparin derivatives and sulfated polysaccharides attenuates H₂O₂ induced islet cell death. Accordingly, it can be seen that heparan sulfate or heparan sulfate mimetics either alone or in combination with known ROS scavengers protects the islet beta cells from reactive oxygen species (ROS) induced cell death. It should also be noted that some heparan sulfate mimetics have heparanase inhibitory activity but the heparan sulfate structural requirements for heparanase inhibition are very different from those required for maintaining beta cell viability and inducing ROS resistance (Table 3).

### Heparan Sulfate Depletion Occurs During Islet Preparation for Transplantation

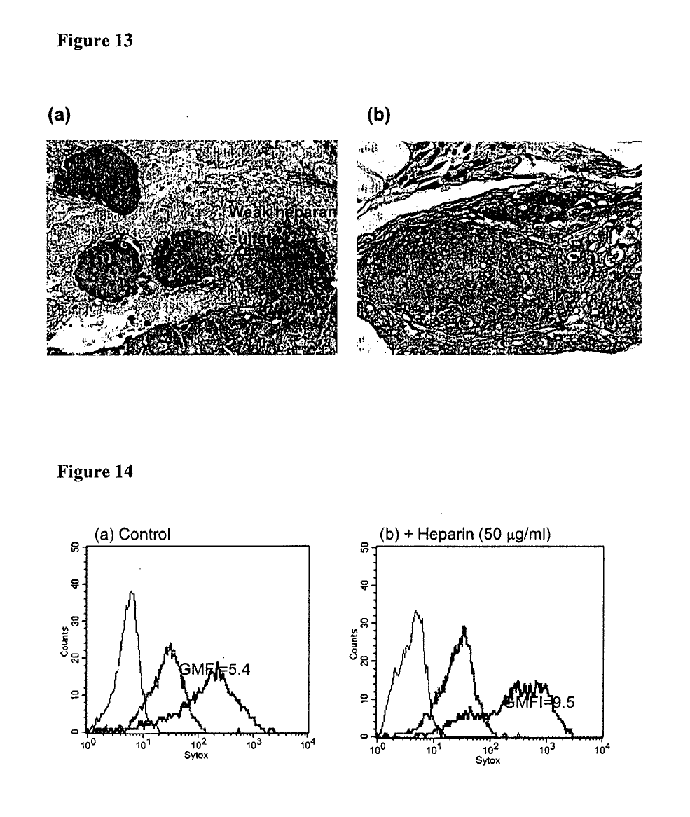

Transplantation of pancreatic islets is a well established therapeutic approach for treating Type I diabetes in animals and patients. However, recovery of fully functional islets is crucial if successful transplantation is to occur, irrespective of the problems associated with the recipient's immune response against the allograft. Based on the data described herein, preservation of intra-islet heparan sulfate during islet isolation is an important factor in ensuring that normal islet function is retained. In fact, examination of mouse islets following isolation revealed that they were substantially and highly significantly depleted (˜60%) of heparan sulfate (P<0.0001, FIG. 12b and histogram) when compared with islets in situ in the pancreas (FIG. 12a and histogram). In fact, following isotransplantation intra-islet heparan sulfate was still depleted 3 days after transplantation but returned to normal levels 7 days post-transplant (FIG. 13). Furthermore, inclusion of heparin in the islet isolation medium resulted in the isolated islets containing almost a 2-fold higher content of insulin (FIG. 14). Thus, heparan sulfate replacement during islet isolation does appear to preserve the functional activity of the islet beta cells indicating that the treatment improves the functional status of beta cells. In addition, islets prepared from mouse donors pretreated with the free radical chemical scavenger butylated hydroxyanisole (BHA; 120 mg/kg i.p.) and isolated in vitro in the presence of the chemical free radical scavenger dimethylthiourea (DMTU; 50 mM) showed increased Alcian blue staining for heparan sulfate. These observations indicate that a combination of heparan sulfate and free radical scavengers could be used to preserve beta cell heparan sulfate during islet isolation.

### Heparan Sulfate Replacement Therapy

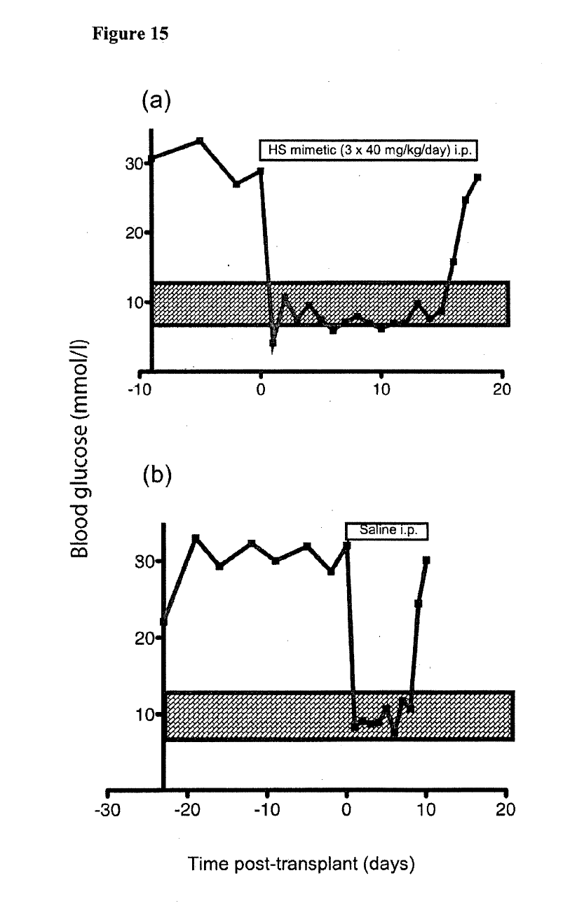

The administration of heparan sulfate mimetics to islet allograft recipients is expected to preserve beta cell function and/or prolong allograft survival. The anticoagulant activity of heparin precludes its prolonged use in vivo but it is possible to obtain heparin derivatives with negligible anticoagulant activity and that possess islet protective properties, e.g., peroxidolysis-glycol split (3 kDa) heparin and other heparin derivatives (Table 3). Indeed treatment of mice with peroxidolysis-glycol split (3 kDa) heparin prevents the acute rejection of islet allografts in experimentally-induced diabetic mice, prolongs graft function and re-establishes normoglycemia in the recipient (FIG. 15).

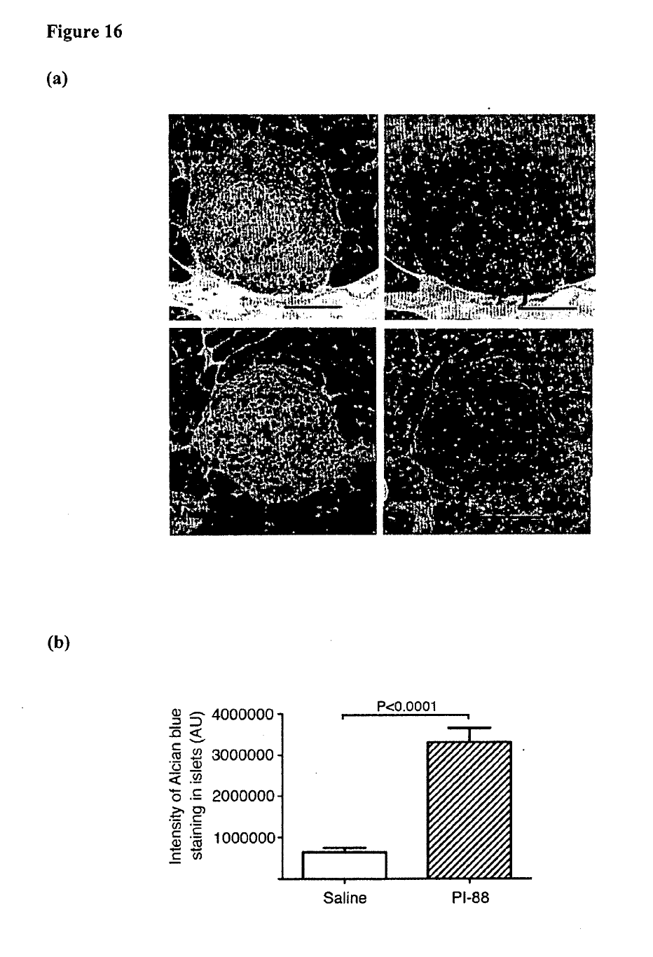

Pre-diabetic. NOD mice that receive a prolonged treatment with the heparan sulfate mimetic PI-88 maintain intra-islet heparan sulfate compared to control, saline treated, pre-diabetic NOD mice. For example FIG. 16 illustrates clear evidence of dramatic loss (˜5-fold) of intra-islet heparan sulfate in the control mice compared to the PI-88 treated mice with abundant intra-islet heparan sulfate being present.

While not being bound by any theory it is postulated that this occurs by directly replacing lost heparan sulfate. Accordingly, administration of heparan sulfate or heparan sulfate mimetics may result in the maintenance of intra-islet heparan sulfate.

The experimental data described herein indicates that intra-islet heparan sulfate is essential for pancreatic islet beta cell function and survival. While not being bound by any hypothesis one mechanism by which this may occur is by protection of beta cells from free radical damage. For example, the heparan sulfate may act as a ‘sink’ for reactive oxygen species or play an indirect role in protecting the beta cells.

Loss of heparan sulfate from islets, either immune mediated via the action of heparanase in Type I diabetes or metabolic stress-induced in Type II diabetes, is a common factor that links the two forms of diabetes. Accordingly, heparan sulfate replacement therapy, either using a heparan sulfate mimetic or derivative represents a treatment for any disease associated with heparan sulfate loss, in particular Type I and Type II diabetes.

### Heparan Sulfate Mimetics

Heparan sulfate is a glycosaminoglycan expressed as a proteoglycan on most cell surfaces and is a component of the extracellular matrix surrounding mammalian cells. Additionally to providing structural integrity heparan sulfate proteoglycans act as a storage site for a variety of heparan sulfate (HS)-binding proteins, including growth factors and chemokines. The polysaccharide component of heparan sulfate is composed of alternating glucuronic acid and N-acetylglucosamine units which may be modified by O-sulfation at various positions, N-deacetylation, and N-sulfation of N-acetylglucosamine residues as well as C-5 epimerization of glucuronic acid to iduronic acid. This structural diversity is further enhanced by variation in chain length of the glycosaminoglycan. The epimerized or sulfated disaccharides in HS are concentrated in “hot spots” along the molecular backbone and separated by flexible spacers of low sulfation, rather than being evenly distributed throughout the polysaccharide chain. HS is known to interact with a wide range of functionally diverse proteins, such as growth factors, cytokines, chemokines, proteases, lipases, and cell adhesion molecules and can regulate the function of HS-binding proteins.

Heparan sulfate mimetics include any molecule which can perform at least one biological function of heparan sulfate, including those referred to above. Several heparan sulfate mimetics have also been isolated or synthesized that are very effective at maintaining beta cell viability and rendering the beta cells resistant to reactive oxygen species (ROS) (Table 3). These mimetics include sulfated oligosaccharides, such as PI-88, maltohexaose sulfate and maltopentaose sulfate, glycol-split porcine mucosal heparin and other glycol split variants (i.e., de-N-sulfated, re-N-acetylated; de-6-sulfated), glycol split low molecular weight heparin (3 kDa) generated by peroxidolysis, and certain sulfated polysaccharides (i.e., dextran sulfate and pentosan polysulfate).

Heparan sulfate mimetics useful in the present invention are selected from the group comprising glycan mimetics, sulfomannan oligosaccharide, sulfated polysaccharides, sulfated oligosaccharides, phosphorothioate oligodeoxynucleotides, sulfated malto-oligosaccharides, phosphosulfomannans, glycol-split heparin, sulfated spaced oligosaccharides, sulfated linked cyclitols, sulfated oligomers of glycamino acids, pseudodisaccharides, suramin and suramin analogues.

The sulfated polysaccharide is selected from the group comprising heparin, λ-carrageenan, κ-carrageenan, fucoidan, pentosan polysulfate, 6-O-carboxymethyl chitin III, laminarin sulfate, calcium spirulan and dextran sulfate.

An example of a sulfated linked cyclitol may be selected from compounds represented by formulae 1 and 3. The compound represented by formula 2 is the starting reagent for making the cyclitol.

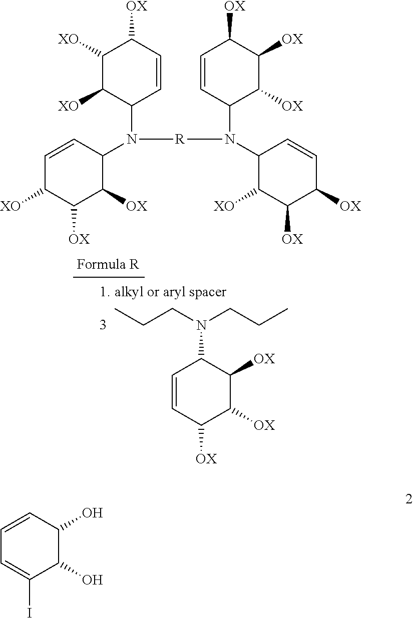

In formulae 1 and 3 X may be SO₃Na or H.

An Example of a phosphosulfomannan heparan sulfate mimetic is formulae 4 below. In one embodiment the heparan sulfate mimetic may be maltohexaose sulfate, O-α-D-Glucopyranosyl-{(1→4)—O-α-D-glucopyranosyl}4-(1→4)-D-glucopyranose sulfate (C₃₆H₆₂O₃₅S).

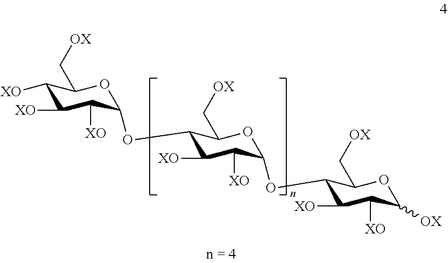

In formulae 4 X may be SO₃Na or H.

Examples of glycan mimetics include low-molecular weight anionic glycan mimetic.

The low molecular weight anionic glycan mimetic may be selected from the group consisting of: a monosaccharide, a disaccharide, an oligosaccharide, a cyclic oligosaccharide (for example a cyclodextrin), a cyclitol, an arylene urea comprising one or more anionic residues, a pseudo sugar, and mixtures thereof.

In one embodiment, the monosaccharide is a sulfated monosaccharide, the disaccharide is a sulfated disaccharide and the oligosaccharide is a sulfated oligosaccharide.

In one embodiment, the monosaccharide may be a ring-opened monosaccharide, the disaccharide may be a ring-opened disaccharide, and the oligosaccharide may be a ring-opened oligosaccharide.

In another embodiment, the low molecular weight anionic glycan mimetic is a monosaccharide, disaccharide, oligosaccharide, ring-opened monosaccharide, ring-opened disaccharide or ring-opened oligosaccharide having the following structural formula:

A-(B)ₐ

wherein a is an integer between 0, 1, 2, 3, 4, 5, 6, 7, 8 and 9; A is selected from the group consisting of: a diose, a triose, a tetraose, a pentose, a hexose, a heptose, an octose and a nonose, and each independent B is selected from the group consisting of: a diose, a triose, a tetraose, a pentose, a hexose, a heptose, an octose and a nonose;

wherein A and B, and where a is an integer of 2 or greater, B and B, linked via a group selected from: —O—(CH₂)ₓ—O—, —O—, —OCH₂—, —NH—, —S—, —NR(CH₂)ₓ—Ar—(CH₂)ₓNR₁—, —NR(CH₂)ₓNR₁—, —O(CH₂)ₓ—Ar—(CH₂)ₓO—, —C(O)—N(R₂)—(CH₂)ₓ—N(R₂)—C(O)—, —N(R₂)—C(O)—Ar—(CH₂)ₓ—Ar—C(O)—N(R₂)— and —N(R₂)—(CH₂)ₓ—N(R₂)—; R, R₁ and R₂ are selected from the group consisting of: hydrogen, alkyl, aryl, heteroaryl and C(O)-alkyl;

x is an integer between 0 and 10;

wherein A and B may be substituted with a functional group selected from the group consisting of: alkyl, alkenyl, aryl, halo, heteroaryl, an amide derivative such as —NHCOCH₃—, alkoxy such as —OCH₃—, —O— and —OH;

and wherein said diose, triose, tetraose, pentose, hexose, heptose, octose and nonose may be sulfated, phosphorylated or carboxylated.

In an embodiment of the first aspect, A and each B are independently selected from the group consisting of a pentose, a hexose and a heptose, and are linked via a group selected from: —O—(CH₂)ₓ—O—, —O—, —OCH₂—, —NR(CH₂)ₓ—Ar—(CH₂)ₓNR₁—, —O(CH₂)ₓ—Ar—(CH₂)ₓO—, —C(O)—N(R₂)—(CH₂)ₓ—N(R₂)—C(O)—, —N(R₂)—C(O)—Ar—(CH₂)ₓ—Ar—C(O)—N(R₂)—, and R, R₁ and R₂ are selected from the group consisting of: hydrogen, acetyl and alkyl, and x is an integer between 1, 2, 3, 4, 5 and 6.

In another embodiment of the first aspect, the hexose may be selected from the group consisting of: glucose, galactose, mannose, fructose, fucose, and idose, and the pentose may be xylose.

In a further embodiment of the first aspect, the low molecular weight anionic glycan mimetic is a cyclitol having the following structural formula:

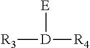

wherein:

D is selected from the group consisting of: N, CH, O, S, or a linker selected from —CO—NH-G-NH—CO—, —NH—CO-G-CO—NH—, —NH-G-NH—, —O-G-O—;

G is selected from the group consisting of alkylene and arylene;

R₃ is a 4-, 5-, or 6-membered carbocyclic ring that is saturated or unsaturated, wherein the ring comprises at least one sulfate group, at least one carboxylate group or at least one phosphate group.

R₄ is selected from the group consisting of: a 4-, 5-, or 6-membered carbocyclic ring that is saturated or unsaturated, wherein the ring comprises at least one sulfate group, at least one carboxylate group or at least one phosphate group, hydrogen, aryl and alkyl; E is selected from the group consisting of: hydrogen, alkyl, aryl, —B—C(R₅)(R₆) and acetate;

B is selected from the group consisting of: —(CH₂)ₓ—, —CH₂ArCH₂—, —CH₂CH(OH)CH₂—, —(CH₂)ₓ—Ar—(CH₂)ₓ—, wherein the B group may optionally comprise one or more sulfate groups, one or more carboxylate groups or one or more phosphate groups.

R₅ and R₆ are independently selected from the group consisting of: 4-, 5-, or 6-membered carbocyclic ring that is saturated or unsaturated, hydrogen, aryl and alkyl, wherein R₅ and/or R₆ may comprise one or more sulfate groups, one or more carboxylate groups or one or more phosphate groups, and x is an integer between 0 and 10.

In one embodiment, B is selected from the group consisting of: —(CH₂)ₓ—, wherein x is an integer between 2, 3, 4, 5, 6, 7, 8, 9 and 10, CH₂ArCH₂ and CH₂CH(OSO₃H)CH₂.

In an alternative embodiment, R₃, R₄, R₅ and R₆ may be independently selected from the following:

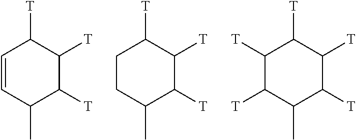

wherein T is independently selected from the group consisting of: SO₃H, SO₃⁻, COOH, COO⁻, OPO₃H and OPO₃⁻.

In a further embodiment of the first aspect, the low molecular weight anionic glycan mimetic is an arylene urea of the following formula:

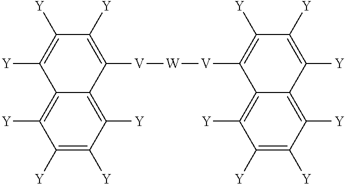

wherein each Y is independently selected from the group consisting of: SO₃H, SO₃⁻, hydrogen, alkyl, halo, phenyl, an amide derivative, —NHCOCH₃, —O—, —OCH₃, COOH, COO⁻, OPO₃H and OPO₃⁻.

each V is independently selected from the group consisting of —(NHC(O)Ph)_{z}-, (CH₂)ᵤ and phenyl;

### W is —NH—C(O)—NH—;

u and z may independently of each other be an integer between 0, 1, 2, 3, 4, 5, 6, 7, 8, 9 and 10.

In one embodiment, the arylene urea may be suramin, or a salt thereof.

In another embodiment of the invention, the glycosaminoglycan mimetic may be a sulfated cyclic oligosaccharide, wherein the oligosaccharide is cyclodextrin.

In a further embodiment of the invention the low molecular weight anionic glycan mimetic is aprosulate.

### Synthesis of Compounds

Low molecular weight anionic glycan mimetics for use in the methods of the invention may be purchased or prepared by methods known to those skilled in the art.

Sulfated saccharide compounds used in the methods and compositions of the invention may be prepared by sulfation of a corresponding monosaccharide, disaccharide or oligosaccharide also by methods known to those skilled in the art. For example, the saccharide compound may be treated with a sulfating agent such as pyridine-sulfur trioxide complex in the presence of an appropriate solvent as follows:

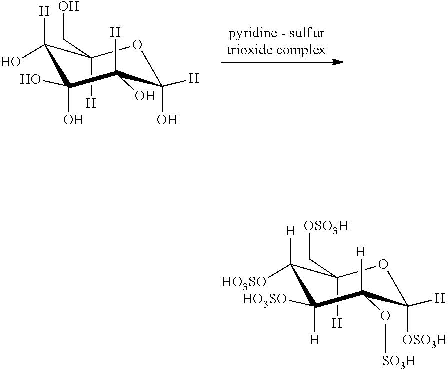

In one aspect, the low molecular anionic glycan mimetic may be a mixture of compounds obtained by reaction of a monosaccharide, disaccharide or oligosaccharide with pyridine-sulfur trioxide complex.

The low molecular weight anionic glycan mimetics may have one or more sulfate groups present. These sulfate groups may react with various bases to form salts. The sulfated compounds are stable when in the form of a salt. The sulfated compounds in a free form may be derived from a salt thereof by utilizing a cation-exchange resin such as Dowex 50W-X8. Optionally, a salt can be subjected to conventional ion-exchange to convert it into any one of various other desirable salts.

The oligosaccharides that are sulfated may be naturally occurring products, for example raffinose, stachyose or cyclodextrins. Alternatively, the oligosaccharides may be prepared by enzymatic or chemical degradation of naturally occurring polysaccharides, followed by subsequent chemical modification.

Other low molecular weight anionic glycan mimetics useful in the methods and compositions of the invention include the following:

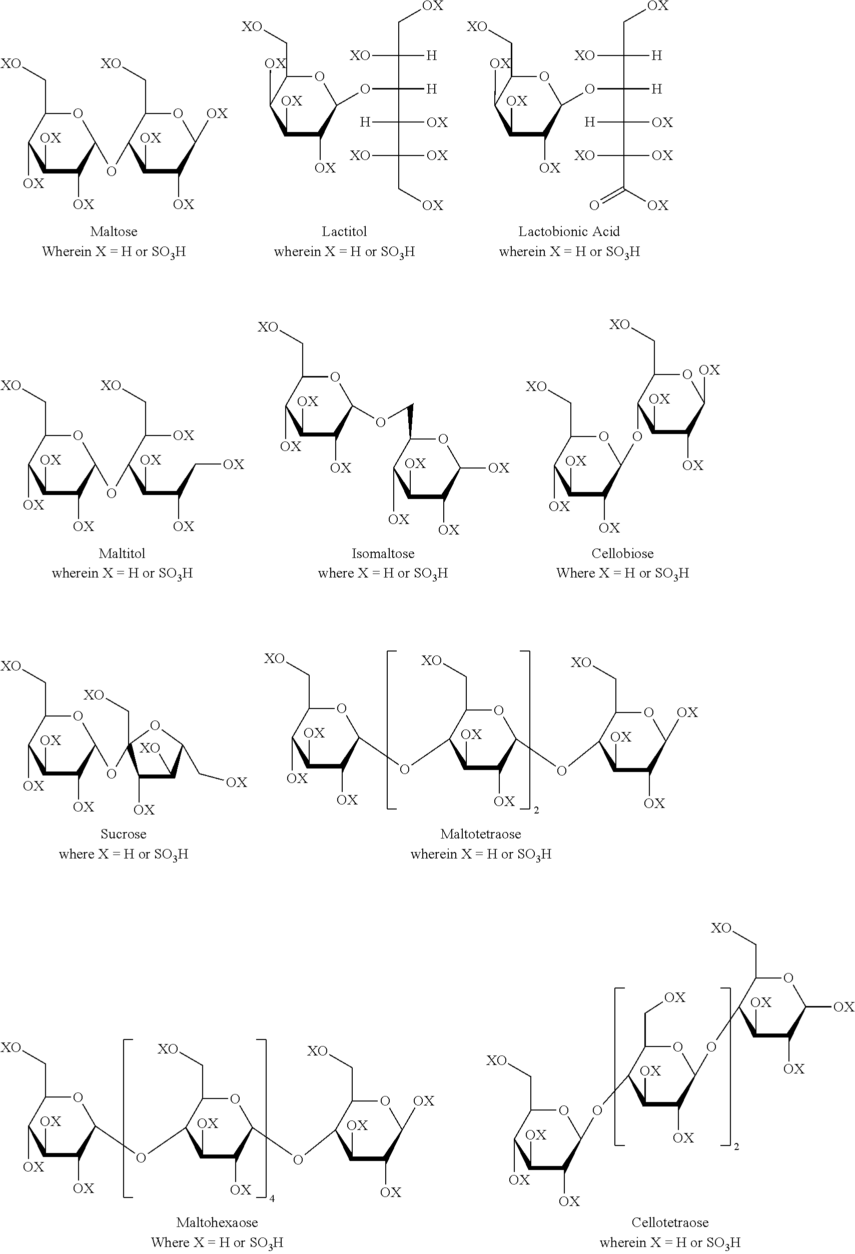

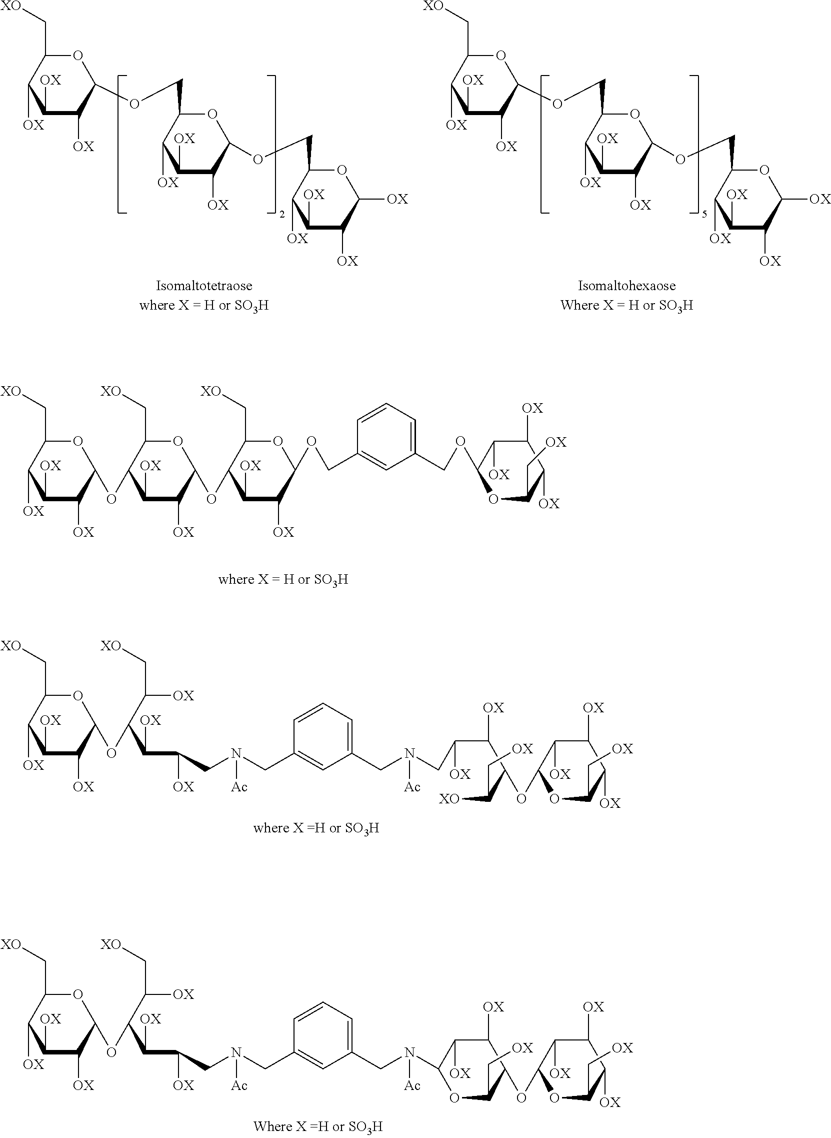

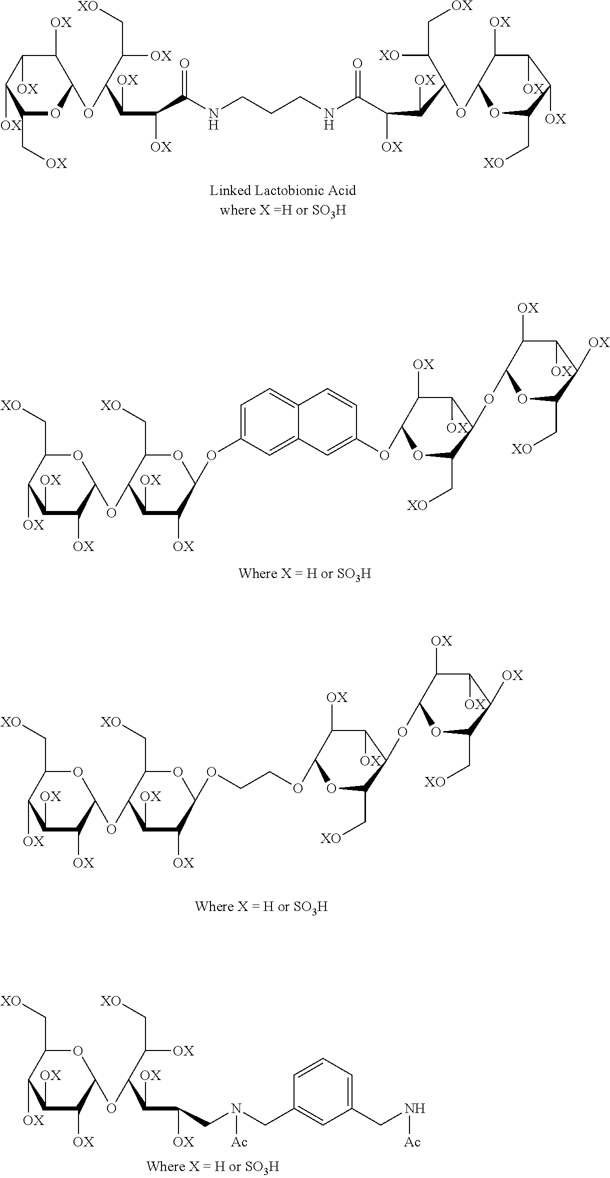

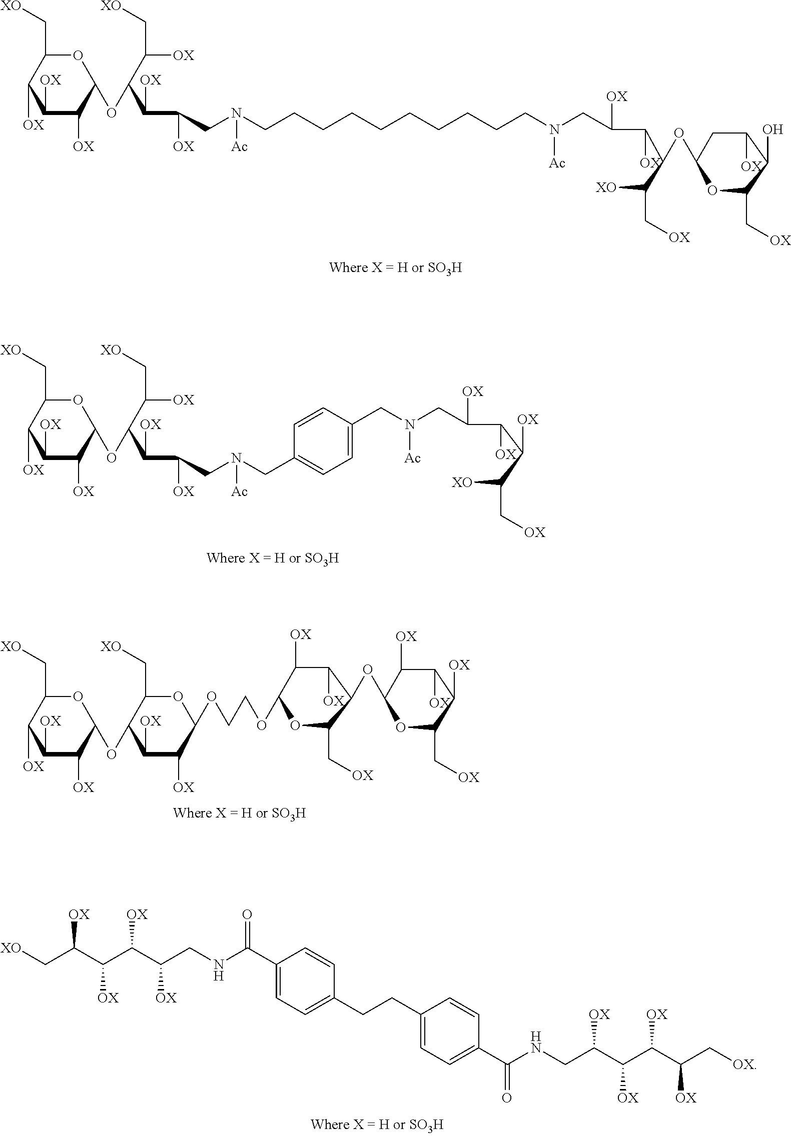

Heparin derivatives useful in the invention as heparan sulfate mimetics may be obtained by “Glycol splitting” of heparin by oxidation with periodate and subsequent reduction with sodium borohydride. Glycol split heparins may be prepared by exhaustive periodate oxidation and borohydride reduction of heparin or N-acetyl heparins with or without prior partial 2-O-desulfation. In one embodiment the heparan sulfate mimetic is peroxidolysis-glycol split (3 kDa) heparin.

### PEGylation of Heparan Sulfate

Unfavorable pharmacokinetics such as a short half-life may decrease the effect of an otherwise effective compound in the treatment of a disease or condition. For example with lower molecular weight polypeptide, carbohydrate or polysaccharide compounds, physiological clearance mechanisms such as renal filtration may make the maintenance of therapeutic levels of such compounds difficult due to the requirement for high frequency dosing.

One solution to an undesirably short serum half-life of a therapeutic compound is to covalently attach a molecule to increase the half-life. It has been shown that attachment of polymers to polypeptides may increase their half-lives. Attachment of therapeutic agents to polymers may also increase aqueous solubility, stability during storage and reduce immunogenicity.

The half-life of the heparan sulfate or heparan sulfate mimetics of the present invention may be increased by linkage to a polymer such as a polyethylene glycol polymer (PEG). The PEG may be linked through any available functionality using methods known in the art. It is preferred that the PEG be linked at only one position in order to minimize any disruption of the activity of the heparan sulfate or heparan sulfate mimetics and to produce a pharmacologically uniform product. Non-limiting examples of functional groups on either the PEG or heparan sulfate or heparan sulfate mimetics which can be used to form such linkages include amine and carboxy groups, thiol groups such as in cysteine residues, aldehydes and ketones, and hydroxy groups as can be found in polysaccharides and in serine, threonine, tyrosine, hydroxyproline and hydroxylysine residues.

An aldehyde functionality useful for conjugating the heparan sulfate or heparan sulfate mimetic to PEG may be generated by sodium periodate oxidation of the saccharide subunits of the heparan sulfate or heparan sulfate mimetic or may be indigenous to the heparan sulfate or heparan sulfate mimetic. The aldehyde functionality can then be coupled to an activated PEG containing a hydrazide or semicarbazide functionality to form a hydrazone or semicarbazone linkage. Hydrazide-containing polymers are commercially available, and can be synthesized, if necessary, using standard techniques.

In a preferred embodiment the heparan sulfate or heparan sulfate mimetic is PEGylated using PEG hydrazide for example by mixing a solution of the two components together and heating to about 37° C. until the reaction is substantially complete. Excess of the polymer hydrazide is typically used to increase the yield of conjugate. By way of example detailed determination of reaction conditions for both oxidation and coupling is set forth in Geoghegan et. al. (1992).

Alternatively the reducing end of a saccharide subunit of heparan sulfate or heparan sulfate mimetic may be used to reduce an amine group of a polymer to result in a secondary amine bond with the C1 carbon atom at the reducing end of the saccharide and the amine group of the polymer. In a preferred embodiment the polymer may be a PEG polymer.

“PEGylated” refers to the covalent attachment of at least one molecule of polyethylene glycol to a biologically active molecule. The average molecular weight of the reactant PEG is preferably between about 500 and about 100,000 daltons, more preferably between about 20,000 and about 60,000 daltons, and most preferably between about 15,000 and about 40,000 daltons. The method of attachment is not critical, but in preferred embodiments does not alter, or only minimally alters, the activity of the biologically active molecule. PEGylation typically results in an increase in half-life.

PEG is typically a linear polymer with terminal hydroxyl groups of the general formula HO—CH₂CH₂—(CH₂CH₂O)n-CH₂CH₂—OH, where n is from about 5 to about 4000. The terminal H may be substituted with a protective group such as an alkyl or aryl group. Preferably, PEG has at least one hydroxy group, more preferably it is a terminal hydroxy group. It is this hydroxy group which is preferably activated to react with a range of conjugates. There are many forms of PEG and PEG derivatives useful in the invention which are known in the art.

The PEG molecule attached to heparan sulfate or heparan sulfate mimetics in the present invention is not limited to a particular type. The average molecular weight of PEG is preferably from 500-100,000 daltons, more preferably from 20,000-60,000 daltons and even more preferably from 20,000-40,000 daltons. The PEG may be linear or branched.

Heparan sulfates of the invention may exist as proteoglycans or otherwise contain a peptide portion, for example as conjugates to a peptide. The peptide portions of these compounds may be PEGylated by covalently linking at least one PEG polymer to the heparan sulfate or heparan sulfate mimetic.

A variety of methods are known in the art to covalently conjugate PEGs to peptides (for example see, Roberts, M. et al. (2002)). One method for preparing the PEGylated heparan sulfate or heparan sulfate mimetics of the present invention is to use PEG-maleimide to attach PEG to a thiol group on the heparan sulfate or heparan sulfate mimetic. The introduction of a thiol functionality may be achieved by addition of a cysteine residue into the peptide portion described above. A thiol functionality may also be introduced onto the side-chain of the peptide portion by for example acylation of the lysine ε-amino group by a thiol-containing acid.

PEGylation may utilise “Michael addition” (the nucleophilic addition of a carbanion to an alpha or beta unsaturated carbonyl compound) to form a stable thioether linker. This highly specific reaction occurs under mild conditions in the presence of other functional groups. PEG maleimide may also be used as a reactive polymer for preparing PEGylated heparan sulfate or heparan sulfate mimetics, preferably using a molar excess of a thiol-containing heparan sulfate of heparan sulfate mimetic relative to PEG maleimide to drive the reaction to completion. The reactions are typically performed between pH 4.0 and 9.0 at room temperature for between about 1 to 40 hours. Excess of non-PEGylated thiol-containing heparan sulfate or heparan sulfate mimetic is readily separated from the PEGylated product by conventional separation methods. Cysteine PEGylation may be performed using PEG maleimide or bifurcated PEG maleimide. A preferred PEG is a 20 kilodalton linear methoxy PEG maleimide.

PEGylated heparan sulfate or heparan sulfate mimetics of the present invention have an in vitro biological activity that is at least 0.5% that of the corresponding non-PEGylated heparan sulfate or heparan sulfate mimetics. Although some PEGylated heparan sulfate or heparan sulfate mimetics compounds of the invention may have biological activity lower than that of the corresponding non-PEGylated heparan sulfate or heparan sulfate mimetics, this decreased activity is compensated by the compound's extended half-life and/or lower clearance value.

### Heparan Sulfate Albumin Conjugates

Another solution to an undesirably short serum half-life of heparan sulfate is to covalently attach a protein molecule, for example albumin, to increase the half-life. It has been shown that attachment of albumin to a therapeutic compound increases the half-life of that compound. Attachment of heparin sulfate to albumin may also increase aqueous solubility, stability during storage and reduce immunogenicity.

Methods for conjugating molecules to proteins such as albumin are known in the art for example Kratz F, (2008). In one embodiment the heparin sulfate may be attached to a maleimide group using methods known in the art. Albumin is known to contain a reactive sulfhydryl which can react with a maleimide group and thus covalently attach a maleimide carrying molecule to albumin. Methods for attaching maleimide carrying molecules to proteins and for attaching maleimide groups to molecules are known in the art, for example Hermanson, G. T. (2008), Karim, A. S. (1995).

In another embodiment the heparan sulfate may be attached to a peptide, antibody or fragment thereof with affinity for albumin using standard methods. The nature of the peptides or antibodies or fragments thereof having albumin affinity are known in the art, for example as described in Nguyen A, et. al. (2006); Dennis M S, et. al. (2002).

### Heparan Sulfate Lipidylation

The half-life of a heparan sulfate may be increased by lipidylation. Lipidylation is known in the art to comprise the covalent attachment of a lipid or fatty acid to a molecule such as a protein. In the context of the present invention lipidylation of heparan sulfate is expected to increase the half-life of the heparan sulfate. A lipid or fatty acid may be attached to heparan sulfate either directly or via a protein, peptide or synthetic linker by methods known in the art which may include those described in Bartholomew M. Sefton et. al. (1987).

A lipid or fatty acid in this context comprises a hydrocarbon backbone of fatty acids (excluding the terminal acidic group) and typically contains 2 to 40 carbon atoms. The fatty acids for use in the present invention may for example contain between about 6 and about 40 carbon atoms, more preferably between about 10 and about 30 carbon atoms, or between about 15 and about 25 carbon atoms. It will be appreciated that fatty acid chain length may be selected on the basis of the intended use of the product and required circulating half-life. Fatty acids may be saturated or unsaturated or polyunsaturate. Suitable fatty acids may be selected from the group comprising, n-dodecanoate (C₁₂, laurate), n-tetradecanoate (C₁₄, myristate), n-hexadecanoate (C₁₆, palmitate), n-octadecanoate (C₁₈, stearate), n-eicosanoate (C₂₀, arachidate), n-docosanoate (C₂₂, behenate), n-tetracosanoate (C₂₄), n-triacontanoate (C₃₀), n-tetracontanoate (C₄₀), cis-Δ⁹-octadecanoate (C₁₈, oleate) and all cis-Δ^{5,8,11,14}-eicosatetraenoate (C₂₀, arachidonate).

### Compositions, Dosages and Routes of Administration

Heparan sulfate for use in the present invention may be administered as compositions either therapeutically or preventively. In a therapeutic application, compositions are administered to a subject already suffering from a disease (e.g. early after disease onset), in an amount sufficient to resolve or partially arrest the disease and/or its complications or to improve the survival of transplanted islets in patients. Heparan sulfate for use in the present invention may be applied to a preparation of islet beta cells, for example, an in vitro preparation of islet beta cells. The composition should provide a quantity of the compound or agent sufficient to effectively treat the subject.

In general, suitable compositions may be prepared according to methods which are known to those of ordinary skill in the art and accordingly may include a pharmaceutically acceptable carrier, diluent and/or adjuvant.

Methods for preparing administrable compositions are apparent to those skilled in the art, and are described in more detail in, for example, Remington's Pharmaceutical Science, 15th ed., Mack Publishing Company, Easton, Pa., hereby incorporated by reference in its entirety.

The heparan sulfate may be present as pharmaceutically acceptable salts. By “pharmaceutically acceptable salt”, it is meant those salts which, within the scope of sound medical judgement, are suitable for use in contact with tissues of humans and lower animals without the undue toxicity, irritation, allergic response and the like, and are commensurate with a reasonable benefit/risk ratio. Pharmaceutically acceptable salts are well known in the art.

The therapeutically effective amount of heparan sulfate disclosed herein for any particular subject will depend upon a variety of factors including: the disorder being treated and the severity of the disorder; activity of the compositions employed; the age, body weight, general health, sex and diet of the patient; the time of administration; the route of administration; the rate of sequestration of the compositions; the duration of the treatment; drugs used in combination or coincidental with the treatment, together with other related factors well known in medicine.

One skilled in the art would be able, by routine experimentation, to determine an effective, non-toxic amount of the components of the formulations which would be to required to treat applicable to achieve the desired outcome of the methods of the invention.

Generally, an effective dosage of heparan sulfate is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage of heparan sulfate may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Further, it will be apparent to one of ordinary skill in the art that the optimal quantity and spacing of individual dosages will be determined by the nature and extent of the condition being treated, the form, route and site of administration, and the nature of the particular individual being treated. Also, such optimum conditions can be determined by conventional techniques. In some therapeutic applications, the treatment would be for the duration of the disease state.

It will also be apparent to one of ordinary skill in the art that the optimal course of treatment, such as, the number of doses of the composition given per day for a defined number of days, can be ascertained by those skilled in the art using conventional course of treatment determination tests.

In general, suitable compositions may be prepared according to methods which are known to those of ordinary skill in the art and accordingly may include a pharmaceutically acceptable carrier, diluent and/or adjuvant.

Convenient modes of administration include injection (subcutaneous, intravenous, etc.), oral administration, intranasal, inhalation, transdermal application, or rectal administration. Depending on the route of administration, the formulation and/or compound may be coated with a material to protect the compound from the action of enzymes, acids and other natural conditions which may inactivate the therapeutic activity of the compound. The compound may also be administered parenterally or intraperitoneally.

Dispersions of compounds may also be prepared in glycerol, liquid polyethylene glycols, and mixtures thereof and in oils. Under ordinary conditions of storage and use, pharmaceutical preparations may contain a preservative to prevent the growth of microorganisms.

Pharmaceutical compositions suitable for injection include sterile aqueous solutions (where water soluble) or dispersions and sterile powders for the extemporaneous preparation of sterile injectable solutions or dispersions. Ideally, the composition is stable under the conditions of manufacture and storage and may include a preservative to stabilise the composition against the contaminating action of microorganisms such as bacteria and fungi.

In one embodiment of the invention, the compound(s) may be administered orally, for example, with an inert diluent or an assimilable edible carrier. The compound(s) and other ingredients may also be enclosed in a hard or soft shell gelatin capsule, compressed into tablets, or incorporated directly into an individual's diet. For oral therapeutic administration, the compound(s) may be incorporated with excipients and used in the form of ingestible tablets, buccal tablets, troches, capsules, elixirs, suspensions, syrups, wafers, and the like. Suitably, such compositions and preparations may contain at least 1% by weight of active compound. The percentage of the anionic glycan mimetic in pharmaceutical compositions and preparations may, of course, be varied and, for example, may conveniently range from about 2% to about 90%, about 5% to about 80%, about 10% to about 75%, about 15% to about 65%; about 20% to about 60%, about 25% to about 50%, about 30% to about 45%, or about 35% to about 45%, of the weight of the dosage unit. The amount of compound in therapeutically useful compositions is such that a suitable dosage will be obtained.

In another embodiment of the invention, the heparan sulfate may be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The to preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which are incorporated herein by reference.

In a further embodiment of the invention, the heparan sulfate may be administered in an aerosol form (such as liquid or powder) suitable for administration by inhalation, such as by intranasal inhalation or oral inhalation.

The use of such media and agents for pharmaceutically active substances is well known in the art. Except insofar as any conventional media or agent is incompatible with the compound, use thereof in the therapeutic compositions and methods of treatment and prophylaxis is contemplated. Supplementary active compounds may also be incorporated into the compositions according to the present invention. It is especially advantageous to formulate parenteral compositions in dosage unit form for ease of administration and uniformity of dosage. “Dosage unit form” as used herein refers to physically discrete units suited as unitary dosages for the individual to be treated; each unit containing a predetermined quantity of compound(s) is calculated to produce the desired therapeutic effect in association with the required pharmaceutical carrier. The compound(s) may be formulated for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

In one embodiment, the carrier may be an orally administrable carrier.

Another form of a pharmaceutical composition is a dosage form formulated as enterically coated granules, tablets or capsules suitable for oral administration.

Also included in the scope of this invention are delayed release formulations.

Heparan sulfate may also be administered in the form of a “prodrug”. A prodrug is an inactive form of a compound which is transformed in vivo to the active form. Suitable prodrugs include esters, phosphonate esters etc, of the active form of the compound.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

In one embodiment, the compound may be administered by injection. In the case of injectable solutions, the carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by including various anti-bacterial and/or anti-fungal agents. Suitable agents are well known to those skilled in the art and include, for example, parabens, chlorobutanol, phenol, benzyl alcohol, ascorbic acid, thiomerosal, and the like. In many cases, it may be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminium monostearate and gelatin.

Sterile injectable solutions can be prepared by incorporating the compound in the required amount in an appropriate solvent with one or a combination of ingredients enumerated above, as required, followed by filtered sterilisation. Generally, dispersions are prepared by incorporating the analogue into a sterile vehicle which contains a basic dispersion medium and the required other ingredients from those enumerated above.

Tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin or a flavouring agent such as peppermint, oil of wintergreen, or cherry flavouring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the analogue, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the analogue can be incorporated into sustained-release preparations and formulations.

The pharmaceutical compositions may further include a suitable buffer to minimise acid hydrolysis. Suitable buffer agent agents are well known to those skilled in the art and include, but are not limited to, phosphates, citrates, carbonates and mixtures thereof.

Single or multiple administrations of the pharmaceutical compositions according to the invention may be carried out. One skilled in the art would be able, by routine experimentation, to determine effective, non-toxic dosage levels of the compound and/or composition of the invention and an administration pattern which would be suitable for treating the diseases and/or infections to which the compounds and compositions are applicable.

Further, it will be apparent to one of ordinary skill in the art that the optimal course of treatment, such as the number of doses of the compound or composition of the invention given per day for a defined number of days, can be ascertained using convention course of treatment determination tests.

### Combination Regimens

Therapeutic advantages may be realised through combination regimens. Those skilled in the art will appreciate that the heparan sulfate disclosed herein may be administered as part of a combination therapy approach to the treatment of Type I and/or Type II diabetes; In combination therapy the respective agents may be administered simultaneously, or sequentially in any order. When administered sequentially, it may be preferred that the components be administered by the same route.

Alternatively, the components may be formulated together in a single dosage unit as a combination product. Suitable agents which may be used in combination with the compositions of the present invention will be known to those of ordinary skill in the art.

Methods of treatment according to the present invention may be applied in conjunction with conventional therapy. Conventional therapy may comprise treatment of islets before transplantation (e.g. with high oxygen). Conventional therapy may also comprise administration of ROS scavengers, anti-inflammatory therapy, immunosupression therapy, surgery, or other forms of medical intervention.

Examples of ROS scavengers include melatonin, vitamin. E, vitamin C, methionine, taurine, Superoxide dismutase (SOD), catalase (CAT), and glutathione peroxidase (GPX), L-ergothioneine N-Acetyl Cysteine (NAC), vitamin A, beta-carotene, retinol, catechins, epicatechins, epigallocatechin-3-gallate, flavonoids, L-ergothioneine, idebenone, selenium, heme oxygenase-1, reduced glutathione (GSH), resveratrol, Tiron (4,5-dihydroxy-1,3-benzenedisulfonic acid), Tempol (4-hydroxy-2,2,6,6-tetramethylpiperydine-1-oxyl), dimethylthiourea (DMTU) and butylated hydroxyanisole (BHA).

Examples of anti-inflammatory agents include steroids, corticosteroids, COX-2 inhibitors, non-steroidal anti-inflammatory agents (NSAIDs), aspirin or any combination thereof. The non-steroidal anti-inflammatory agent may be selected from the group comprising ibuprofen, naproxen, fenbufen, fenprofen, flurbiprofen, ketoprofen, dexketoprofen, tiaprofenic acid, azapropazone, diclofenac, aceclofenac, diflunasil, etodolac, indometacin, ketorolac, lornoxicam, mefanamic acid, meloxicam, nabumetone, phenylbutazone, piroxicam, rofecoxib, celecoxib, sulindac, tenoxicam, tolfenamic acid or any combination thereof.

Examples of immunosuppressive agents include alemtuzumab, azathioprine, ciclosporin, cyclophosphamide, lefunomide, methotrexate, mycophenolate mofetil, rituximab, sulfasalazine tacrolimus, sirolimus, or any combination thereof.

Compounds and compositions disclosed herein may be administered either therapeutically or preventively. In a therapeutic application, compounds and compositions are administered to a patient already suffering from a condition, in an amount sufficient to cure or at least partially arrest the condition and its symptoms and/or complications. The compound or composition should provide a quantity of the active compound sufficient to effectively treat the patient.

Compounds and compositions disclosed herein may be administered to islets before transplantation.

### Carriers, Diluents, Excipients and Adjuvants

Carriers, diluents, excipients and adjuvants must be “acceptable” in terms of being compatible with the other ingredients of the composition, and not deleterious to the recipient thereof. Such carriers, diluents, excipient and adjuvants may be used for enhancing the integrity and half-life of the compositions of the present invention. These may also be used to enhance or protect the biological activities of the compositions of the present invention.

The language “pharmaceutically acceptable carrier” is intended to include solvents, dispersion media, coatings, anti-bacterial and anti-fungal agents, isotonic and absorption delaying agents, and the like. Examples of pharmaceutically acceptable carriers or diluents are demineralised or distilled water; saline solution; vegetable based oils such as peanut oil, safflower oil, olive oil, cottonseed oil, maize oil, sesame oils, arachis oil or coconut oil; silicone oils, including polysiloxanes, such as methyl polysiloxane, phenyl polysiloxane and methylphenyl polysolpoxane; volatile silicones; mineral oils such as to liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from 10% to 99.9% by weight of the compositions.

The carriers may also include fusion proteins or chemical compounds that are covalently bonded to the compounds of the present invention. Such biological and chemical carriers may be used to enhance the delivery of the compounds to the targets or enhance therapeutic activities of the compounds. Methods for the production of fusion proteins are known in the art and described, for example, in Ausubel et al (In: Current Protocols in Molecular Biology. Wiley Interscience, ISBN 047 150338, 1987) and Sambrook et al (In: Molecular Cloning: A Laboratory Manual, Cold Spring Harbor Laboratories, New York, Third Edition 2001).

The compositions of the invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic parenterally acceptable diluents or carriers can include, Ringer's solution, isotonic saline, phosphate buffered saline, ethanol and 1,2 propylene glycol.

Some examples of suitable carriers, diluents, excipients and/or adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay to disintegration.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin. E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like. The emulsions for oral administration may further comprise one or more emulsifying agents. Suitable emulsifying agents include dispersing agents as exemplified above or natural gums such as guar gum, gum acacia or gum tragacanth.

### Timing of Therapies

Those skilled in the art will appreciate that the compositions may be administered as a single agent or as part of a combination therapy approach to the treatment of diseases to such as Type I and/or Type II diabetes at diagnosis or subsequently thereafter, for example, as follow-up treatment or consolidation therapy as a complement to currently available therapies for such diseases, and as a treatment for transplant recipients. The compositions may also be used as preventative therapies for subjects who are genetically or environmentally predisposed to developing such diseases.

The present invention will now be further described in greater detail by reference to the following specific examples, which should not be construed as in any way limiting the scope of the invention.

### EXAMPLES

### Example 1

### Alcian Blue Staining of Pancreatic Islets

Pancreatic islets, in both mice and humans, contain high levels of the glycosaminoglycan heparan sulfate as indicated by Alcian blue staining (pH5.8, 0.65M MgCl₂) of formalin-fixed pancreas sections (FIG. 1). Like mouse islets, human islets show widespread distribution of heparan sulfate within the islet cell mass.

### Example 2

### Detection of Heparan Sulfate, Collagen XVIII and Syndecan-1 in Pancreatic Islets

Immunohistochemical staining of formalin-fixed mouse islets (post-antigen retrieval with pronase) with HepSS-1 monoclonal antibody specific for heparan sulfate (FIG. 2c) indicates the presence of large amounts of this glycosaminoglycan in pancreatic islets. Islets also contain substantial amounts of collagen type XVIII and syndecan-1 (FIGS. 2a and 2b), well known core proteins for heparan sulfate proteoglycans. Immunostaining for collagen type XVIII and syndecan-1 is observed using appropriate antibodies on formalin-fixed pancreas sections after standard antigen retrieval with citrate buffer.

### Example 3

### Heparan Sulfate Deficiency in Type II Diabetic Mice

Examination of islets from diabetic db/db mice, an obese mouse strain which spontaneously develops Type II diabetes, shows by Alcian blue histochemical staining that the islets contained much less heparan sulfate than islets from db heterozygous control mice that do not develop diabetes. FIG. 3 indicates a substantially reduced level of staining for heparan sulfate in the Type II diabetic db/db islet.

### Example 4

### Heparan Sulfate Maintains Islet Beta Cell Viability and Renders the Cells Resistant to Reactive Oxygen Species

Pancreatic islets were isolated from non-diabetic BALB/c mice and dissociated into a single cell suspension (>90% beta cells) by Dispase digestion. Following 2 days culture in standard tissue culture medium 62% of the beta cells were dead, based on uptake of the fluorescent DNA binding dye, Sytox green (FIG. 4, upper panels). In contrast, if the beta cells were cultured in the presence of heparin (50 μg/ml), highly sulfated heparan sulfate (HS^{hi}, 50 μg/ml) or the heparan sulfate mimetic PI-88 (50 μg/ml) beta cell survival was dramatically enhanced (FIG. 4, upper panels). This remarkably improved viability was confirmed by uptake of Calcein-AM, a fluorescent dye which labels viable and early apoptotic cells, and staining by propidium iodide (PI), a DNA-binding dye that labels dead and apoptotic cells (FIG. 4, lower panels). In the presence of heparin the highly viable Calcein⁺, PI⁻population of beta cells was increased from 25% to 86%, the dead population. (Calcein⁻, PI⁺) reduced from 18% to 1% and the apoptotic population (Calcein⁺, PI⁺) improved from 52% to 6%. As with the Sytox green assay, HS^{hi} and PI-88 at 50 μg/ml were just as effective as heparin at promoting beta cell survival using the Calcein-AM-PI viability assay (FIG. 4, lower panels), although heparin was also equally active at 5 μg/ml (FIG. 5). These effects on increased beta cell viability by the three compounds were found to be highly statistically significant based on multiple experiments (Table 1). Table 1 provides a statistical analysis of the data showing that heparin, highly sulfated heparan sulfate (HS^{hi}) and PI-88 can protect mouse beta cells from culture-induced cell death.

TABLE 1
Heparin, highly sulfated heparan sulfate (HS^{hi}) and PI-88 protect mouse
beta cells from culture-induced cell death

| | % beta cells | | | |
|---|---|---|---|---|
| Treatment | Calcein + PI− | Calcein + PI+ | Calcein − PI+ | Calceln − PI− |
| Control Heparin HS^{hi} PI-88 | 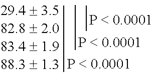 | 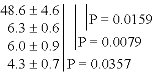 | 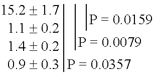 | 7.4 ± 2.2 9.75 ± 1.9 9.0 ± 2.3 6.3 ± 0.9 |

Beta cells were cultured in the presence or absence of 50 μg/ml of heparin, HS^{hi} or PI-88 for 2 days and then Calcein/PI fluorescence staining was used to assess % cell viability (Calcein + PI−), % early apoptotic cells (Calcein + PI+ ), % late apoptotic/dead cells (Calcein − PI+) and cell debris (Calcein − PI−) by flow cytometry; n = 3-5/group.
Student's t test and Mann-Whitney test were used for statistical analyses.

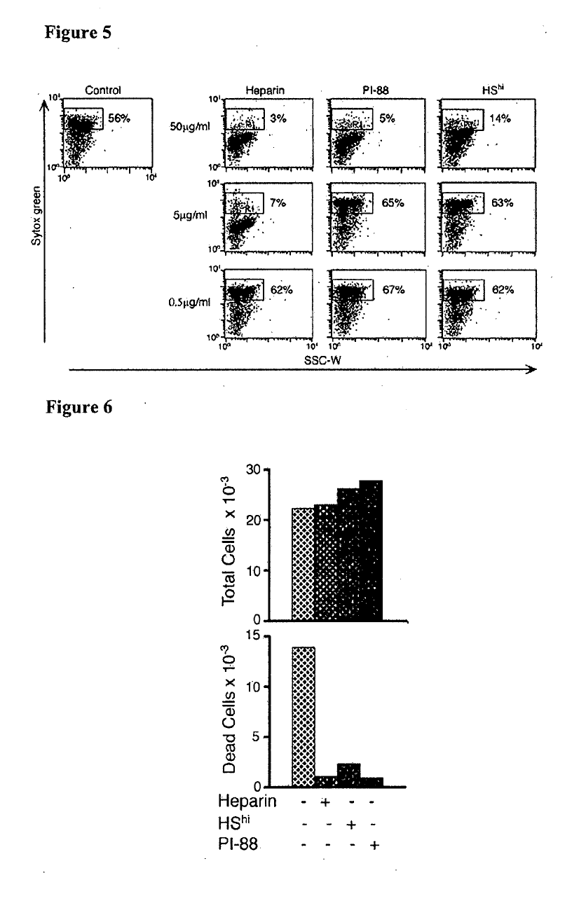

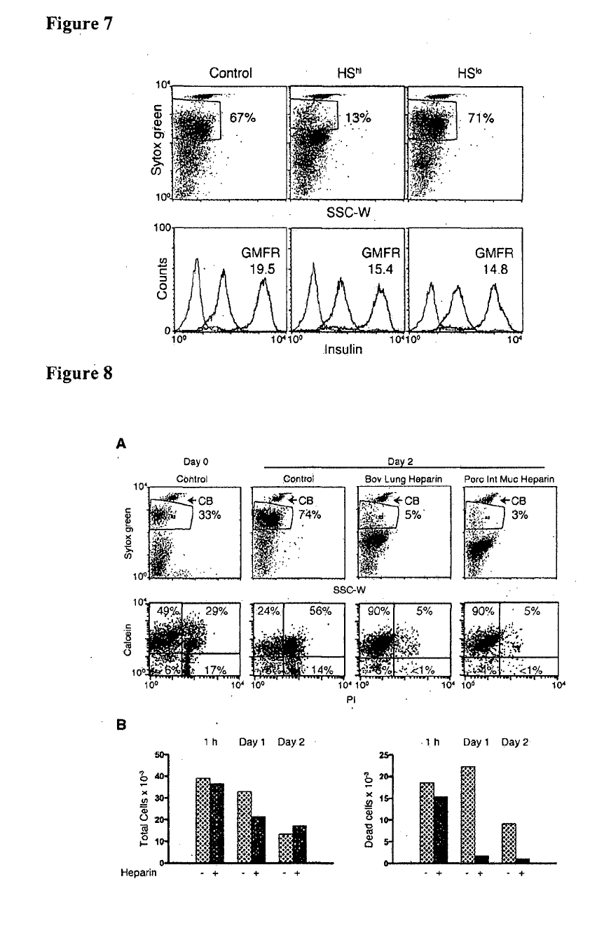

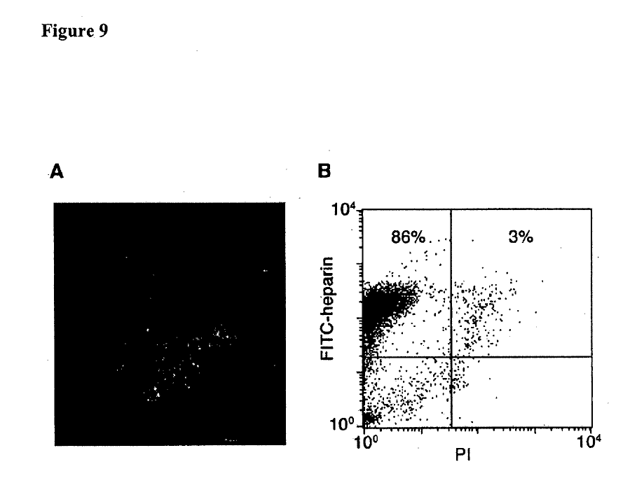

All three compounds at 50 μg/ml also decreased the absolute number of dead beta cells by 6-14 fold compared to controls, despite comparable total cell numbers in the cultures (FIG. 6). In contrast, unlike HS^{hi}, treatment with under-sulfated HS(HS^{lo}) did not protect the beta cells (FIG. 7, upper panels). Heparin/HS^{hi} treatment, however, resulted in little or no change in the insulin content of beta cells. The impact of heparin treatment on beta cell viability was also independent of the source of heparin (FIG. 8a) and, based on viable cell numbers, was not observed after 1 h but did occur after continuous culture with heparin for 1-2 days (FIG. 8b). Studies with FITC-labelled heparin revealed that after 2 days of culture with the fluorescent heparin (50 μg/ml) a large amount of intracellular of FITC-heparin could be detected by confocal microscopy (FIG. 9a), with flow cytometry demonstrating that 89% of beta cells contained high levels of the fluorescent heparin and were highly viable based on PI dye exclusion (FIG. 9b). Collectively these data show that heparin and related compounds (e.g., HS^{hi} and PI-88) can dramatically improve islet beta cell viability following in vitro culture.

Following 2 days culture with heparin (50 μg/ml) the surviving beta cells also became remarkably resistant to hydrogen peroxide-induced cell death (95% viable), whereas freshly isolated beta cells are exquisitely sensitive to peroxide treatment (96.1% cell death) (FIG. 10). Similarly, the sulfated oligosaccharide, PI-88, and highly sulfated heparan sulfate (HS^{hi}), but not lowly sulfated heparan sulfate (HS^{lo}), were able to protect the islet beta cells from peroxide-induced cell death (FIG. 11). These data imply that heparan sulfate protects the islet beta cells from reactive oxygen species (ROS, free radical) induced cell death. Table 2 provides a statistical analysis of the data showing that heparin protects mouse beta cells from culture-induced and reactive oxygen species (ROS)-induced cell death.

TABLE 2
Heparin protects mouse beta cells from culture-induced and ROS-induced cell death

| | | % Sytox +ve beta cells at time after culture | | |
|---|---|---|---|---|
| | Treatment | 1 hour | Day 1 | Day 2 |
| Control Heparin | −H₂O₂ +H₂O₂ −H₂O₂ +H₂O₂ | 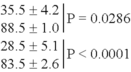 | 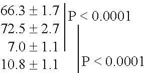 | 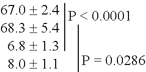 |

Beta cells were cultured in the presence or absence of 50 μg/ml of heparin for 1 h, 1 day or 2 days and then treated with 30% H₂O₂, as a source of ROS, for 5 min. Sytox green uptake was used to assess % cell death by flow cytometry; n = 4/group.

An extensive study of a number of heparan sulfate mimetics revealed that a range of such molecules could maintain beta cell viability and induce resistance to ROS (Table 3). Table 3 compares the ability of a range of compounds (50 μg/ml) to rescue islet beta cells viability following 2 days culture and to induce reactive oxygen species (ROS) resistance in the beta cells. Compounds that retained beta cell viability>85% are highlighted in bold italics. Induction of resistance to ROS is indicated by (+), lack of resistance by (−). All compounds that produced high beta cell viability induced ROS resistance.

TABLE 3
Ability of compounds to rescue β-cell viability, induce ROS resistance and
inhibit heparanase

| | | Viability | Resistance | Hpse |
|---|---|---|---|---|
| Compound | (kDa) | (%) | to H₂O₂ (ROS) | inhibition |
| Heparins | | | | |
| Porcine mucosal heparin | 12.5 | [symbol] | ++++ | +++ |
| decarboxylated | 12.5 | 66 | − | ++* |
| glycol split, | 10 | [symbol] | ++++ | ++++ |
| glycol split, deNS, reNA | 10 | [symbol] | ++++ | ++++ |
| glycol split, de6S | 12.5 | [symbol] | ++++ | +++ |
| glycol split, de2S | 12.5 | 50 | − | ++++* |
| Low Mol Wt Heparin (Enoxaparin) | 3 | 38 | − | − |
| peroxidolysis | 3 | 27 | + | − |
| peroxidolysis-glycol split | 3 | [symbol] | ++++ | +++± |
| nitrous acid-glycol split | 3 | 30 | + | +++±* |
| Sulfated oligosaccharides | | | | |
| PI-88 (20% tet/70% pent) | 2-2.5 | [symbol] | ++++ | +++ |
| Maltohexaose sulfate | 3 | [symbol] | ++++ | +++ |
| Maltopentaose sulfate | 2.5 | [symbol] | ++++ | +++ |
| Maltotetraose sulfate (75% tet/25% pent) | 2 | 39 | − | +++* |
| Bis-lactobionic acid amide (C12 link) | 2 | 64 | − | +++* |
| Other polysaccharides | | | | |
| Dextran sulfate | 5.5 | [symbol] | ++++ | ++ |
| Pentosan PS | 5 | [symbol] | ++++ | ++ |
| HS High S | 12-15 | [symbol] | +++ | ±* |
| HS Low S | 15 | 29 | − | − |
| Chondroitin sulfate A | 20 | 50 | − | ± |
| Chondroitin sulfate B | 30 | 46 | − | − |
| Chondroitin sulfate C | 60 | 32 | − | − |
| Chondroitin sulfate D | ~60 | 32 | − | ± |
| Hyaluronic acid (HA) low MW | 80 | 75 | − | − |
| HA decasaccharide | 2 | 66 | − | − |
| HA | >1 mDa | 31 | − | − |
| Chitosan | 100 | 30 | − | − |
| Fucoidin (F. vesiculosis) | 20 | 80 | − | ++* |

Beta cells were cultured in the presence or absence of 50 μg/ml of the different compounds for 2 days and then treated with 30% H₂O₂, as a source of ROS, for 5 min. Sytox green uptake was used to assess % cell viability by flow cytometry after 2 days culture and after ROS exposure. Those compounds that maintained beta cell viability >85% after 2 days culture are highlighted in bold italics. Note that control untreated beta cell exhibited only 25-30% viability after 2 days culture. The heparanase inhibitory activity of the different compounds was assessed as previously described (Freeman C. and Parish C. R. (1997).
+ to ++++ Ability of compounds to render beta cells resistant to ROS or inhibit heparanase enzymatic activity, with + being lowest and ++++ highest activity.
− Compounds that failed to protect beta cells against ROS or inhibit heparanase. These compounds also usually failed to protect beta cells against culture-induced cell death.
± Very weak protection of beta cells against ROS or heparanase inhibitory activity.
*Compounds that either selectively protect beta cells against ROS or selectively inhibit heparanase.
deNS = de-N-sulfated
de6S = de-6-sulfated
reNA = re-N-acetylated
de2S = de-2-sulfated

It was found that sulfated oligosaccharides, such as maltohexaose sulfate and maltopentaose sulfate, glycol split porcine mucosal heparin and other glycol split variants (i.e., de-N-sulfated, re-N-acetylated; de-6-sulfated), glycol split low molecular weight heparin (3 kDa) generated by peroxidolysis, and certain sulfated polysaccharides (i.e., dextran sulfate and pentosan polysulfate) induced high beta cell viability and ROS resistance (Table 3). However, there were strict structural requirements for such biological activity. Thus, the activity of porcine mucosal heparin was largely lost after decarboxylation but was fully retained following glycol splitting (Table 3). Indeed, de-N-sulfated, re-N-acetylated glycol split heparin and de-6-sulfated glycol split heparin were still highly active whereas de-2-sulfated glycol split heparin exhibited low activity (Table 3). Furthermore, low molecular weight (3 kDa) heparins generated by either peroxidolysis or nitrous acid cleavage were completely inactive, whereas glycol split low molecular weight heparin generated by peroxidolysis became highly active. In contrast, low molecular weight heparin generated by nitrous acid cleavage, when glycol split, remained inactive (Table 3).

The low molecular weight heparins suggested that oligosaccharide chain length strongly influences the ability of compounds to retain beta cell viability and induce ROS resistance. This observation was confirmed with the maltose series of sulfated oligosaccharides, the hexa- and penta-saccharides being highly active whereas the tetrasaccharide (maltotetraose sulfate) was completely inactive. However, chain length was not the only requirement for biological activity as most glycosaminoglycans, except for heparin and HS^{hi}, were inactive (Table 3).

Collectively these data indicate that there are highly specific structural requirements for heparan sulfate mimetics to maintain beta cell viability and protect beta cells from ROS damage. However, many of these active mimetics are much more suitable for clinical use than heparin as they lack many of the other biological activities of heparin, notably anticoagulant properties.

### Example 5

### Ability of Heparan Sulfate Mimetics to Protect Beta Cells from ROS and to Inhibit Heparanase are Unrelated Biological Activities

Previous studies by the inventors have shown that heparan sulfate mimetics can act as heparanase inhibitors and protect mice from the induction of Type I diabetes, heparanase allowing autoreactive T lymphocytes to enter the islets and destroy intra-islet heparan sulfate (WO2008/046162). However, the ability of heparan sulfate mimetics to maintain beta cell viability and render beta cells resistant to ROS is a totally different function of these molecules unrelated to their heparanase inhibitory activity. In fact, several of the compounds listed in Table 3 (marked with asterisks) selectively inhibited these two different biological processes. For example, decarboxylated heparin, glycol split de-2-sulfated heparin, nitrous acid cleaved-glycol split LMW heparin, maltotetraose sulfate, bis-lactobionic acid amide and fucoidan were all strong to very strong heparanase inhibitors but were essentially unable to induce islet beta cells to become ROS resistance. Conversely, highly sulfated heparan sulfate is a very poor heparanase inhibitor but is a potent inducer of ROS resistance in islet beta cells. These data also imply that the heparan sulfate structural requirements for heparanase inhibition are very different from those required for maintaining beta cell viability and inducing ROS resistance.

### Example 6

### Heparan Sulfate Loss in Transplanted Pancreatic Islets

Examination of mouse islets following isolation revealed that they were substantially (˜60%) and highly significantly (P<0.0001) depleted of heparan sulfate (FIG. 12b and histogram) when compared with islets in situ in the pancreas (FIG. 12a). This deficiency in intra-islet heparan sulfate persisted for at least 3 days after transplantation of the islets into histocompatible recipients, normal heparan sulfate levels only being regained 7 days post-transplantation (FIG. 13). Inclusion of heparin (50 μg/ml) in the islet isolation medium improved the insulin content of the islet beta cells 2-fold (FIG. 14).

### Example 7

### Heparan Sulfate Mimetics Prolong Islet Allograft Survival

In order to assess the efficacy of heparan sulfate mimetics in prolonging islet allograft survival, C57BL/6J (H-2^{b}) mice were made diabetic by treatment with alloxan and their diabetic state reversed by the transplantation of allogeneic CBA (H-2^{k}) islets. The transplanted allogeneic islets are typically only able to restore blood glucose to normoglycemic levels 1-8 days post-transplantation but are then rejected (FIG. 15b). On the other hand, administration of a heparan sulfate mimetic (peroxidolysis-glycol split (3 kDa) heparin) allowed the graft-induced restoration of normal blood glucose levels to persist for an additional 7 days (FIG. 15a). The heparan sulfate mimetic was administered thrice daily in order to inhibit the extremely vigorous allograft rejection reaction observed with H-2^{k} to H-2^{b} transplants.

### Example 8

### Heparan Sulfate Mimetics Preserve Islet Heparan Sulfate

Treatment of pre-diabetic NOD mice with 10 mg/kg/day i.p. of the heparan sulfate mimetic PI-88 preserved the heparan sulfate content of pre-diabetic islets, as measured by Alcian. Blue staining, compared to saline treated control mice which exhibited substantial loss of islet heparan sulfate (FIG. 16a). In fact, quantification of heparan sulfate staining revealed that islets from PI-88-treated mice contained ˜5-fold higher levels of heparan sulfate than islets from saline treated control NOD mice, a difference that was highly statistically significant (P<0.0001) (FIG. 16b)

### Example 9

### Production of Heparin Derivatives

### Low Molecular Weight Heparin Derivatives:

Low molecular weight heparin (sodium salt) from porcine intestinal mucosa, average mol wt ˜3,000 (cat no. H3400) was obtained from Sigma-Aldrich and was prepared by depolymerization by peroxidolysis (free-radical induced cleavage).

### Nitrous Acid Cleavage of Heparin:

Heparin was cleaved by nitrous acid degradation at pH 4 (Reaction A) adapted from Lindahl (1973) and Lagunoff and Warren (1962). Briefly, 200 mg of heparin was dissolved in 2 ml of water and an equal volume of 0.48 M sodium nitrite in 3.6 M acetic acid was added and the mixture stirred for 9 min at room temperature. The pH was raised to 7 with NaOH, the solution dialyzed and reduced with 200 mg sodium borohydride for 4 h. The mixture was acidified with HCl, dialyzed and lyophilized to give 3 kDa heparin.

### 6-O-desulfated Heparins:

6-O-desulfated heparins were prepared according to Matsuo et al (1993) by reaction with N,O-bis(trimethylsilyl)acetamide without N-desulfation occurring. Briefly, heparin (200 mg) was converted into its pyridinium salt and dissolved in pyridine (20 ml). After addition of 4 ml of N-methyl-N-(trimethylsilyl)trifluoroacetamide, the solution was heated for 4 h at 80° C. to yield the 6-O-desulfated heparin which was dialysed against water and lyophilized.

### Carboxyl Reduced Heparins:

Heparin (250 mg in 50 ml of water) was carboxyl reduced by an adaptation of the method of Karamanous et. al. (1988) by adding N-(3-dimethylaminopropyl)-N-ethylcarbodiimide (EDC, 1 mg) at room temperature, followed by acidification with 10 ml of 0.04 M HCl and stirring for 1 h. Reduction of the carbodiimide ester was accomplished with fresh 2 M NaBH₄ (200 mL in two portions) at 50° C. for 2 h. Excess NaBH₄ was decomposed with HOAc, the solution dialyzed against water and lyophilized.

### N-acetylated Heparins:

N-acetylated heparins were prepared by N-desulfation under solvolytic conditions by the method of Nagasawa et. al. (1979). Briefly the pyridinium salt of heparin was stirred at 20° C. in Me₂SO:water (9:1) for 8 h to obtain N-desulfated intermediates which upon N-acetylation with acetic anhydride in alkaline aqueous medium (0.5 M NaHCO₃, 4° C., 2 h) by the method of Levvy and McAllan (1959).

### Glycol-Split Heparins:

Glycol-split heparins were prepared by exhaustive periodate oxidation and borohydride reduction of heparin by the method of Casu et al (2002). Briefly, 250-mg of heparin was dissolved in 6 ml of H₂O, and 6 ml of 0.2M NaIO₄ was added to the solution which was stirred at 4° C. for 16 h in the dark. The reaction was stopped by adding 2 ml of ethylene glycol, and the solution dialyzed for 16 h. Solid sodium borohydride (60 mg) was added to the heparin solution in several portions while stirring. After 3 h the pH was adjusted to 4 with 0.1 M HCl, and the solution neutralized with 0.1 M NaOH. After dialysis against water, the final product was lyophilized.

### 2-O-desulfated Heparins:

2-O-desulfated heparin was prepared according to Jaseja et al (1989). Briefly, heparin (500 mg) was dissolved in 500 ml of 0.1 M NaOH and the solution was frozen and lyophilized. The residue was dissolved in 500 ml of distilled water, neutralized with HCl and dialyzed against water. The product was isolated by lyophilization.

### REFERENCES

Geoghegan and Stroh, (1992), Bioconjugate Chem. 3:138-146
Roberts, M. et al. (2002) Advanced Drug Delivery Reviews, 54:459-476.
Kratz F, (2008) J Controlled Release 132 171-183.
Hermanson, G. T. (2008) Bioconjugate Techniques 2nd edition, Academic Press, New York,
Karim, A. S. (1995) Maleimide-mediated protein conjugates of a nucleoside triphosphate gamma-S and an internucleotide phosphorothioate diester. Nucl. Acid. Res. 23(11), 2037-2040.
Nguyen. A, Reyes A E 2nd, Zhang M, McDonald P, Wong W L, Damico L A, Dennis M S. Protein Eng Des Sel. (2006) July; 19(7):291-7. Epub 2006 Apr. 18;
Dennis M S, Zhang M, Meng Y G, Kadkhodayan M, Kirchhofer D, Combs D, Damico L A. J Biol Chem. (2002) September 20; 277(38):35035-43. Epub 2002 Jul. 15.
Bartholomew M. Sefton and Janice E. Buss (1987) The Journal of Cell Biology, Vol. 104, No. 6, pp. 1449-1453
Casu B, Guerrini, M, Naggi, A, Perez, M, Toni, G, Ribatti, D, Carminati P, Giannini G, Penco S, Pisano C, Belleri M, Rusnati M and Presta M (2002) Short heparin sequences spaced by glycol-split uronate residues are antagonists of fibroblast growth factor 2 and angiogenesis inhibitors. Biochemistry 41 10519-10528
Freeman C and Parish C R. (1997) A rapid quantitative assay for the detection of mammalian heparanase activity. Biochem J. 325, 1341-1350.
Jaseja, M., Rej, R. N., Sauriol, F. and Perlin, A. S. (1989) Novel regio- and stereo-selective modifications of heparin in alkaline solution. Nuclear magnetic resonance spectroscopic evidence Can. J. Chem. 67, 1449-1456
Karamanos, N. K. Hjerpe, A. Tsegenidis, T. Engfeldt B. and Antonopoulos, C. A. (1988) Determination of iduronic acid and glucuronic acid in glycosaminoglycans after stoichiometric reduction and depolymerization using high-performance liquid chromatography and ultraviolet detection. Anal. Biochem. 172. 410-419
Lagunoff, D and Warren, G. (1962) Determination of 2-deoxy-2-sulfoaminohexose content of mucopolysaccharides Arch Biochem Biophys, 99: 396-400
Levvy, G. A., and McAllan, A. (1959) The N-acetylation and estimation of hexosamines. Biochem. J. 73, 127-159
Lindahl U, Bäckström G, Jansson L, Hallén A. (1973) Biosynthesis of heparin. II. Formation of sulfamino groups. J Biol Chem. 248: 7234-41.
Matsuo, M., Takano, R., Kamei-Hayashi, K., and Hara, S. (1993) A novel regioselective desulfation of polysaccharide sulfates: Specific 6-O-desulfation with N,O-bis(trimethylsilyl)acetamide. Carbohydr. Res. 241, 209-215
Nagasawa, K., Inoue, Y., and Kamata, T. (1977) Solvolytic desulfation of glycosaminoglycuronan sulfates with dimethyl sulfoxide containing water or methanol. Carbohydr. Res. 58, 47-55

1. A method for inhibiting oxidative damage of islet beta cells in a subject comprising administering to the subject a therapeutically effective amount of heparan sulfate.

2. A method for inhibiting oxidative damage of islet beta cells comprising contacting said beta cells with heparan sulfate.

3. A method of treating diabetes comprising administering to a subject in need thereof a therapeutically effective amount of heparan sulfate.

4. The method claim 3 wherein the diabetes is Type-I or Type-II diabetes.

5. A method of treating an autoimmune condition comprising administering to a subject in need thereof a therapeutically effective amount of heparan sulfate.

6. The method of claim 5 wherein the autoimmune condition may be selected from the group comprising Type 1 diabetic insulitis, rejection of pancreatic islet transplant or a combination thereof.

7. A method of preserving beta-cell function comprising administering to a subject in need thereof a therapeutically effective amount of heparan sulfate.

8. The method of claim 7 wherein the beta-cell is a transplanted beta-cell.

9. A method of preserving beta-cell function in isolated islets comprising pretreating the islets with a therapeutically effective amount of heparan sulfate prior to transplantation into a patient.

10. A method of treating or preventing the rejection of a transplant comprising administering to a subject in need thereof a therapeutically effective amount of heparan sulfate.

11. A method for reducing the level of immunosuppressive therapy associated with transplantation comprising administering to a subject in need thereof a therapeutically effective amount of heparan sulfate.

12. The method of claim 10 or claim 11 wherein the transplant is a pancreatic islet transplant.

13. A method for preserving endogenous heparan sulfate comprising administering to a subject a therapeutically effective amount of heparan sulfate.

14. The method of any one of claims 1 to 13 further comprising administration of a reactive oxygen species scavenger in combination with the heparan sulfate.

15. The method of claim 14 wherein the reactive oxygen species scavenger is selected from the group consisting of melatonin, vitamin E, vitamin C, methionine, taurine, Superoxide dismutase (SOD), catalase (CAT), and glutathione peroxidase (GPX), L-ergothioneine N-Acetyl Cysteine (NAC), vitamin A, beta-carotene, retinol, catechins, epicatechins, epigallocatechin-3-gallate, flavonoids, L-ergothioneine, idebenone, selenium, heme oxygenase-1, reduced glutathione (GSH), resveratrol, Tiron (4,5-dihydroxy-1,3-benzenedisulfonic acid, Tempol (4-hydroxy-2,2,6,6-tetramethylpiperydine-1-oxyl), dimethylthiourea (DMTU) and butylated hydroxyanisole (BHA).

16. Use of heparan sulfate for the preparation of a medicament for preserving beta-cell function.

17. Use of heparan sulfate for the preparation of a medicament for treatment of diabetes.

18. The use of claim 16 wherein the diabetes is Type-I or Type-II diabetes.

19. Use of heparan sulfate for the preparation of a medicament for treatment of transplant rejection.

20. Use of heparan sulfate for the preparation of a medicament for inhibiting the rejection of a transplant in a subject.

21. Use of heparan sulfate for the preparation of a medicament for reducing the level of immunosuppressive therapy associated with transplantation.

22. The use of any one of claims 19 to 21 wherein the transplant is a pancreatic islet transplant.

23. The use of any one of claims 16 to 22 further comprising the use of a reactive oxygen species scavenger in combination with the heparan sulfate in the preparation of the medicament.

24. The use of claim 23 wherein the reactive oxygen species scavenger is selected from the group comprising melatonin, vitamin E, vitamin C, methionine, taurine, Superoxide dismutase (SOD), catalase (CAT), glutathione peroxidase (GPX), L-ergothioneine N-Acetyl Cysteine (NAC), vitamin A, beta-carotene, retinol, catechins, epicatechins, epigallocatechin-3-gallate, flavonoids, L-ergothioneine, idebenone, selenium, heme oxygenase-1, reduced glutathione (GSH), resveratrol, Tiron (4,5-dihydroxy-1,3-benzenedisulfonic acid), Tempol (4-hydroxy-2,2,6,6-tetramethylpiperydine-1-oxyl), dimethylthiourea (DMTU) and butylated hydroxyanisole (BHA).

25. The method of any one of claims 1 to 15 or the use of any one of claims 16 to 24 wherein the heparan sulfate is maltohexaose sulfate.

26. The method of any one of claims 1 to 15 or the use of any one of claims 16 to 24 wherein the heparan sulfate is covalently bound to a molecule to increase the half-life of the heparan sulfate.

27. The method or use of claim 26 wherein the covalently bound heparan sulfate is PEGylated.

28. The method or use of claim 26 wherein covalently bound heparan sulfate is peroxidolysis-glycol split (3 kDa) heparin.
